(19) United States
(12) Patent Application Publication
(10) Pub. No.: US 2022/0218971 A1
(43) Pub. Date: Jul. 14, 2022

(54) NANOPROJECTION DEVICES AS WELL AS METHODS OF MAKING AND USING SUCH DEVICES

(71) Applicant: CORNELL UNIVERSITY, Ithaca, NY (US)

(72) Inventors: Ankur SINGH, Ithaca, NY (US); Sungwoong KIM, Ithaca, NY (US); Brian RUDD, Ithaca, NY (US)

(21) Appl. No.: 17/609,883

(22) PCT Filed: May 11, 2020
(86) PCT No.: PCT/US2020/032369

§ 371 Date: Nov. 9, 2021

Related U.S. Application Data
(60) Provisional application No. 62/846,219, filed on May 10, 2019.

(51) Int. Cl. A61M 37/00

(52) U.S. Cl. CPC A61M 37/0015; B82B 3/0076

(57) ABSTRACT

The present application relates to a silicon nanoprojection device comprising a substrate having a surface and one or more nanoprojection structures having a proximal end attached to said substrate and extending away from the surface of the substrate to a distal end. The one or more nanoprojection structures either have a configuration which tapers narrowingly from the proximal end to the distal end or have an ionic coating. Also disclosed are methods of making and using the silicon nanoprojection device.

This application claims the priority benefit of U.S. Provisional Patent Application Ser. No. 62/846,219, filed May 10, 2019, which is hereby incorporated by reference in its entirety.

### FIELD

The present application discloses nanoprojection devices, as well as methods of making and using such devices.

### BACKGROUND

Various methods for the delivery of biomolecules such as nucleic acids, gene editing materials, and proteins to the cytoplasm of a target cell cytoplasm are known in the art. These include: (1) microinjection in which DNA is injected directly into the nucleus of cells through fine glass needles; (2) dextran incubation, in which DNA is incubated with an inert carbohydrate polymer (dextran) to which a cationic chemical group (DEAE, for diethylaminoethyl) is attached; (3) calcium phosphate coprecipitation, in which cells efficiently take in DNA in the form of a precipitate with calcium phosphate; (4) electroporation, in which cells are placed in a solution containing DNA and subjected to a brief electrical pulse that causes holes to open transiently in their membranes so that DNA enters through the holes directly into the cytoplasm; (5) liposomal mediated transformation, in which DNA is incorporated into artificial lipid vesicles, liposomes, which fuse with the cell membrane, delivering their contents directly into the cytoplasm; (6) biolistic transformation, in which DNA is absorbed to the surface of gold particles and fired into cells under high pressure using a ballistic device; (7) naked DNA insertion; (8) viral-mediated transformation, in which nucleic acid molecules are introduced into cells using viral vectors (e.g., retroviruses, lentivirus, adenovirus, herpesvirus, and adeno-associated virus vectors); and (9) nanowire mediated delivery, in which biomolecules are permanently or reversibly attached to a nanowire structure via a covalently bound linker and target cells are contacted with the nanowire structures to allow penetration of the nanowires into the cell to deliver the biomolecule into the cell.

Despite the advantages that many of these systems provide, many have serious drawbacks.

For example, electroporation, liposomal mediated transformation, and cationic delivery may result in low delivery efficiency and poor cell viability.

Nanowires, which have been shown to penetrate cells, fail to effectively deliver genetic material and other biomolecules to target cells. Although previous studies have demonstrated nanowire-mediated biomolecule delivery to ex vivo primary immune cells, e.g., bone marrow derived dendritic cells, B cells, dendritic cells, macrophages, natural killer cells, and T cells (Shalek et al., “Nanowire-Mediated Delivery Enables Functional Interrogation of Primary Immune Cells: Application to the Analysis of Chronic Lymphocytic Leukemia,” Nano. Lett. 12(12): 6498-6504 (2012)), such studies are carried out using nanowire arrays which have defects, are subject to manufacturing irregularities, are easily broken, are not reusable, comprise nanowires that are oriented at an angle of between 60 to 90 degrees relative to a substrate surface, do not comprise a safety stop feature, and low uniformity, which results in low transfection efficiencies and suboptimal cell viability.

The present application is directed to overcoming these and other deficiencies in the art.

### SUMMARY

One aspect of the present application relates to a silicon nanoprojection device comprising a substrate having a surface and one or more nanoprojection structures having a proximal end attached to the substrate and extending away from the surface of the substrate to a distal end. The one or more nanoprojection structures have a configuration which tapers narrowingly from the proximal end to the distal end.

Another aspect of the present application relates to a silicon nanoprojection device comprising a substrate having a surface; one or more nanoprojection structures having a proximal end attached to the substrate and extending away from the surface of the substrate to a distal end; and an ionic coating on the one or more nanoprojection structures.

Yet another aspect of the present application relates to a method of making a nanoprojection device. This method involves providing a silicon monolithic structure and carrying out a series of nanofabrication steps on the silicon monolithic structure to form one or more nanoprojection structures having a proximal end attached to a surface of a substrate and extending away from the surface of the substrate to a distal end. The one or more nanoprojection structures have a configuration which tapers narrowingly from the proximal end to the distal end.

A further aspect of the present application relates to a method for delivering a biomolecule to a target cell. This method involves providing a silicon nanoprojection device according to the present application and contacting one or more target cells with the one or more nanoprojection structures of the silicon nanoprojection device, so that the one or more nanoprojection structures extend into the one or more target cells.

Also disclosed are one or more modified target cells produced according to the methods described herein.

Another aspect of the present application relates to a method of treating a subject with a modified cell. This method involves selecting a subject in need of treatment with a modified cell and administering one or more modified target cells as described herein to treat the selected subject.

The examples presented here demonstrate the ability of uniformly designed nanoprojection array devices to make even contacts with target cells in a controlled manner to efficiently deliver large quantities of a selected biomolecule to the cytoplasm of a target cell, without compromising cell viability. This is in contrast to prior art that utilizes uncharged silicon surfaces or biomolecule tethering using direct covalent bonding to silicon.

As described herein, the use of functionalized nanoprojection arrays to perturb target cells represents a promising, minimally destructive strategy for intracellular delivery of target biomolecules by allowing for effector specific manipulation with negligible effects on cell survival and function. Furthermore, the effective delivery of cell effectors can regulate cellular behavior, expressing desired phenotypes, and activating cells to express specific markers. This platform may enable the manufacture of therapies at a large scale. In addition, prior art has not shown the ability to deliver multiple types of biomolecules simultaneously.

### BRIEF DESCRIPTION OF THE DRAWINGS

FIGS. 1A-1I are schematic illustrations showing the fabrication of a silicon nanoprojection device having a surface and one or more nanoprojection structures having a proximal end attached to the substrate and extending away from the surface of the substrate to a distal end, where the one or more nanoprojection structures have a configuration which tapers narrowingly from the proximal end to the distal end. As shown in this schematic, a silicon wafer is deposited with a silicon dioxide (SiO₂) etching mask layer (FIG. 1A) and fine patterns of arrays are developed using deep UV photolithography (FIG. 1B). The fine pattern are transferred to the SiO₂ etching mask layer via dry etching (FIG. 1C). Deep silicon reactive-ion etching (RIE) is carried out to produce high aspect ratio nanoprojection structures extending away from the surface of the substrate (FIG. 1D). Tapering of the nanoprojection structures is carried out using a soft dry etching process (FIG. 1E) to produce nanoprojections having, e.g., sub-10 nm tips. The tapered nanoprojection structure surface is functionalized with a strongly charged capturing layers, e.g., by covalently attaching a modifier (e.g., silane-PEG-hydroxysulfosuccinimide (NETS) moieties) (FIG. 1F), which may then be conjugated with an ionic polymer (e.g., polyethyleneimine (PEI, branched, 25 kDa)) (FIG. 1G) for electrostatic biomolecule complexation (FIG. 1H). Target cells (e.g., T cells) are then contacted with the silicon nanoprojection device to induce intracellular delivery of a target biomolecule (FIG. 1I).

FIGS. 2A-2C are scanning electron microscopy (SEM) images of high aspect ratio nanoprojection structures corresponding to FIG. 1D (FIG. 2A), tapered nanoprojection structures corresponding to FIG. 1E (FIG. 2B), and T cells cultured on tapered nanoprojection structures corresponding to FIG. 1I (FIG. 2C).

FIGS. 3A-3G demonstrate intracellular delivery procedures carried out using the bare tapered nanoprojection array of FIG. 1E. FIG. 3A is a schematic illustration of a bare tapered nanoprojection array (top panel) coated with a target biomolecule (e.g., miRNA29-FITC or FITC-Dextran)(middle panel), and contacted with a target cell (e.g., a CD8⁺ T cell) (bottom panel). FIGS. 3B-3E are dot plots showing the delivery efficiency of FITC-Dextran (3,000-5,000 g/mol) alone (FIG. 3B); FITC-Dextran (3,000-5,000 g/mol) coated onto a bare tapered nanoprojection array (FIG. 3C); miRNA29-FITC alone (FIG. 3D); and miRNA29-FITC coated onto a bare tapered nanoprojection array (FIG. 3E). FIGS. 3F-3G are graphs showing the mean fluorescence intensity of TBET (FIG. 3F) and EOMES (FIG. 3G) following bare nanoprojection-mediated delivery of miRNA29-FITC alone (black bars) or deposited onto a bare tapered nanoprojection array (grey bars).

FIGS. 4A-4F demonstrate intracellular delivery of miRNA29-FITC carried out using coated nanoprojection arrays. FIG. 4A shows a schematic illustration of a bare tapered nanoprojection array spin-coated with polyethyleneimine (PEI) (top panel) or vapor-phage coated with 3-(trihydroxysilyl)-1-propanesulfon (bottom panel) prior to miRNA29-FITC deposition. FIG. 4B is a dot plot of naïve T cells used to gate for miRNA29-FTIC. FIGS. 4C-4E are dot plots showing the delivery efficiency of: miRNA29-FITC deposited onto a bare tapered nanoprojection array (FIG. 4C); miRNA29 deposited onto a PEI-coated nanoprojection array corresponding to FIG. 4A (FIG. 3D); and miRNA29 deposited onto the surface of a 3-(trihydroxysilyl)-1-propanesulfon-coated nanoprojection array corresponding to FIG. 4B (FIG. 4E). FIG. 4F is a bar graph showing the delivery efficiency of miRNA29-FITC under the conditions described in FIGS. 4B-4E. *(P≤0.05), One-way ANOVA (Turkey); n=3.

FIGS. 5A-5F demonstrate the dose effect of PEI concentration on target gene expression and cytotoxicity in T cells. FIGS. 5A-5B are graphs showing the expression levels of TBET (FIG. 5A) and EOMES (FIG. 5B) in T cells following delivery of miRNA29-FITC deposited onto a bare tapered nanoprojection array (+miRNA+Nano) or miRNA29-FITC deposited on PEI-coated nanoprojection arrays functionalized with 10 wt % (+miRNA+Nano10PEI), 25 wt % (+miRNA+Nano25PEI), or 50 wt % (+miRNA+Nano50PEI) PEI. FIGS. 5C-5D are dot plots showing T cell viability (FIG. 5C) and transfection efficiency (FIG. 5D) following the delivery of miRNA29-FITC alone. FIGS. 5E-5F are dot plots showing T cell viability (FIG. 5E) and transfection efficiency (FIG. 5F) following the delivery of miRNA29-FITC deposited onto a HPEI-coated (50 wt %) tapered nanoprojection array. *(P≤0.05), One-way ANOVA (Dunnett); n=2.

FIGS. 6A-6F demonstrate that covalent modification of tapered nanoprojection arrays with silane-PEG-NHS modifiers reduces cell toxicity. FIG. 6A is a schematic illustration showing the modification of a tapered silicon nanoprojection device. As shown in this schematic, the silicon nanoprojection device (left panel) is covalently modified with a silane-PEG-NHS modifier (second panel from the left), spin coated with 10 wt % PEI (third panel from the left), and deposited with 1 μM miRNA29-FITC (fourth panel from the left). FIGS. 6B-6D are dot plots showing the strategy (FIG. 6B) used to gate cells evaluated for viability (FIG. 6B) and delivery efficiency (FIG. 6D) following delivery of miRNA-29 deposited onto PEI coated silane-PEG-NHS modified tapered silicon nanoprojection arrays. FIG. 6E is a dot plot showing the delivery efficiency of miRNA29-FITC alone. FIG. 6F is a histogram showing an overlay of miRNA29-FITC delivery carried out under the conditions described in FIG. 6D (dark grey) and FIG. 6E (light grey).

FIGS. 7A-7B are confocal microscopic images of CD8⁺ T cells following intracellular delivery of FITC conjugated RNA molecules for 48 hours. FIG. 7A shows a CD8⁺ T cell treated with negative control (NC)-FITC RNA. FIG. 7B shows a CD8⁺ T cell treated with miRNA29-FITC. Grey around edges of cell: CD8⁺; diffuse grey in center of cell: RNA-FITC.

FIGS. 8A-8B demonstrate the dose effect of miRNA29-FITC concentration on the intracellular delivery efficiency carried out using PEI coated tapered nanoprojection arrays modified with silane-PEG-NHS. FIG. 8A is a histogram showing the intracellular delivery efficiency when T cells were contacted with 10 μM miRNA29-FITC alone (histogram furthest to the left) or complexed with an ionically charged nanoprojection array at the following concentrations: 0.1 μM miRNA29-FITC (second histogram from the left), 0.1 μM miRNA29-FITC (third histogram from the left), 1 μM miRNA29-FITC (fourth histogram from the left), and 10 μM miRNA29-FITC (fifth histogram from the left). FIG. 8B is a graph showing the delivery efficiency of miRNA29-FITC vs. concentration of miRNA29-FITC (μM).

FIGS. 9A-9G demonstrate the delivery efficiency of FITC-conjugated RNA molecules to target cells and the effect of the delivered FITC-conjugated RNA molecules on the expression of transcription factors T-BET and EOMES. FIGS. 9A-9F are dot plots showing the cell viability and delivery efficiency of T cells contacted with miRNA29-FITC alone (FIGS. 9A-9B, respectively), miR29-FITC deposited onto a PEI-coated tapered nanoprojection array modified with silane-PEG-NHS (FIGS. 9C-9D, respectively), and negative control (NC) RNA (NC-FITC) deposited onto a PEI-coated tapered nanoprojection array modified with silane-PEG-NHS (FIGS. 9E-9F). FIG. 9G is a graph showing the expression of TBET and EOMES in T cells contacted with NC-FITC RNA (▪) or miRNA29 FITC (▴) deposited onto PEI-coated tapered nanoprojection arrays modified with silane-PEG-NHS, as compared to control conditions (•).

FIGS. 10A-10G are dot plots showing the co-delivery of two microRNAs using charged tapered nanoprojection arrays. FIGS. 10A-10B show the percentage of mir130 mimic⁺ cells (FIG. 10A) and miR29 antisense oligonucleotide (ASO)⁺ cells (FIG. 10B) following delivery of miR29ASO+miR130 mimic in the absence of a nanoprojection array. FIGS. 10C-10D show the percentage of mir130 mimic⁺ cells (FIG. 10C) and miR29 antisense oligonucleotide (ASO)⁺ cells (FIG. 10D) following delivery of negative control (NC) ASO+ NC-mimic in the presence of a nanoprojection array. FIGS. 10E-10F show the percentage of mir130 mimic⁺ cells (FIG. 10E) and miR29 antisense oligonucleotide (ASO)⁺ cells (FIG. 10F) following delivery of miR29ASO+miR130 mimic in the presence of a nanoprojection array.

FIG. 10G is a bar graph showing the fold change of NC-ASO (left bar), mir-29 ASO (second bar from left), NC-mimic (third bar from left), and mir-130 mimic (fourth bar from left) relative to β-actin.

FIGS. 11A-11E demonstrate the results of a CD8⁺ T cell proliferation test of miRNA29-FITC and negative control miRNA-FITC (NC-FITC). FIGS. 11A-11C are histograms showing the proliferation of T cells treated in the presence of a nanoprojection device+miRNA29-FITC (FIG. 11A), in the presence of a nanoprojection device+NC-FITC (FIG. 11B), and in the absence of a nanoprojection device (FIG. 11C). FIG. 11D is an overlay of the histograms shown in FIGS. 11A-11C. FIG. 11E is a bar graph showing the dilution of proliferation dye in control cells (left bar), cells treated in the presence of a nanoprojection device with NC (middle bar), and cells treated in the presence of a nanoprojection device+miR29.

FIGS. 12A-12E demonstrate the activation markers of CD8⁺ T cells and their different viable cell percentage. FIGS. 12A-12D are histograms showing the expression of CD25⁺ (FIG. 12A), CD69⁺ (FIG. 12B), CD44⁺ (FIG. 12C), and CD62L⁺ (FIG. 12D) in CD8⁺ T cells treated with control, in the presence of a nanoprojection device+NC, or in the presence of a nanoprojection device in the presence of mir29. FIG. 12E is a bar graph showing the results of FIGS. 12A-12D.

FIGS. 13A-13D demonstrate the cytokine production of CD8⁺ T cells and their different viable cell percentage. FIGS. 13A-13C are histograms showing the production of granzyme B (FIG. 13A), TNFα (FIG. 13B), and IFNγ (FIG. 13C) in CD8⁺ T cells treated with control, in the presence of a nanoprojection device+NC, or in the presence of a nanoprojection device in the presence of mir29. FIG. 13D is a bar graph showing the results of FIGS. 13A-13C.

FIGS. 14A-14E demonstrate target expression level of CD8⁺ T cells and their qPCR from the co-delivery of mir29 and mir130. FIGS. 14A-14D are histograms showing the expression of IRF1 (FIG. 14A), CD130 (FIG. 14B), EOMES (FIG. 14C), and T-bet (FIG. 14D) in CD8⁺ T cells treated in the presence of a nanoprojection device+mir29+mir130, as compared to control. FIG. 14E is a bar graph showing the results of FIGS. 14A-14D.

FIGS. 15A-15D compare the CD8⁺ T cell proliferation rate with negative control and co-delivery of mir29 and mir130. FIGS. 15A-15C are histograms showing the proliferation of CD8⁺ T cells treated in the presence of a nanoprojection device+NC (FIG. 15A), a nanoprojection device+29a ASO+130b mim (FIG. 15B), and an overlay of the results seen in FIGS. 15A and 15B (FIG. 15C). FIG. 15D is a bar graph showing the dilution of proliferation dye following treatment of CD8⁺ T cells in the presence of a nanoprojection device+29a ASO+130b min (left bar), in the presence of a nanoprojection device+NC (second bar from left), or control (third bar from the left).

FIGS. 16A-16D show the activation and differentiation of CD8⁺ T cells of negative control and co-delivery of mir29 and mir30. FIGS. 16A-16C are histograms showing the expression of CD69 (FIG. 16A), CD44 (FIG. 16B), and CD62L (FIG. 16C) following treatment of CD8⁺ T cells in the presence of a nanoprojection device+NC as compared to when CD8⁺ T cells were treated with a nanoprojection device+29a ASO+130b mim. FIG. 16D is a bar graph showing the results of FIGS. 16A-16C.

FIGS. 17A-17D show the cytokine production of CD8⁺ T cells treated in the presence of negative control and during co-delivery of mir29+mir130 in the presence of a nanoprojection device. FIGS. 17A-17C are histograms showing the production of IFNγ (FIG. 17A), granzyme B (FIG. 17B), and TNFα (FIG. 17C). FIG. 17D is a bar graph showing the results of FIGS. 17A-17D.

### DETAILED DESCRIPTION

The present application relates to silicon nanoprojection devices, methods of making nanoprojection devices, methods of delivering a biomolecule to a target cell, target cells or preparations of target cells produced according to the disclosed methods, and methods of treating a subjected using the disclosed target cells or preparation of target cells.

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements, or method steps.

The terms “comprising”, “comprises”, and “comprised of” also encompass the term “consisting of”.

Unless defined otherwise, all technical and scientific terms used herein have the same meaning as commonly understood to one of ordinary skill in the art to which this application belongs.

The recitation of numerical ranges by endpoints includes all numbers and fractions subsumed within the respective ranges, as well as the recited endpoints.

One aspect of the present application relates to a silicon nanoprojection device comprising a substrate having a surface and one or more nanoprojection structures having a proximal end attached to the substrate and extending away from the surface of the substrate to a distal end. The one or more nanoprojection structures have a configuration which tapers narrowingly from the proximal end to the distal end.

In some embodiments, the silicon nanoprojection device further comprises an ionic coating over the one or more nanoprojection structures.

Another aspect of the present application relates to a silicon nanoprojection device comprising a substrate having a surface; one or more nanoprojection structures having a proximal end attached to the substrate and extending away from the surface of the substrate to a distal end; and an ionic coating on the one or more nanoprojection structures.

As used herein, the term “nanostructure” refers to a material in the shape of a solid wire or rod (sometimes tapered) having a cross-sectional diameter in the range of 1 nm to 1000 nm. For example, a nanoprojection may have a cross-sectional diameter of 1 nm-1000 nm, 1 nm-900 nm, 1 nm-800 nm, 1 nm-700 nm, 1 nm-600 nm, 1 nm-500 nm, 1 nm-400 nm, 1 nm-300 nm, 1 nm-200 nm, 1 nm-100 nm, 10 nm-1000 nm, 10 nm-900 nm, 10 nm-800 nm, 10 nm-700 nm, 10 nm-600 nm, 10 nm-500 nm, 10 nm-400 nm, 10 nm-300 nm, 10 nm-200 nm, or 10 nm-100 nm. In embodiments, the cross-sectional diameter refers to a longest dimension of a cross-section of a referenced structure, without limiting the cross-section of the referenced structure to a circle. In embodiments, the cross-section of the referenced structure can comprise a circle, an oval, an ovoid, an ellipsoid, a tear-drop shape, an ellipsoidal shape, an oviform shape, or an irregular shape.

As used herein, the terms “nanoprojection structure” or “nanoprojections” refer to a nanowire having a proximal end and a distal end. The cross-sectional diameter of the proximal end and the cross-sectional diameter of the distal end are not equivalent when the nanoprojections are tapered. In such embodiments, the “nanoprojection structure” or “nanoprojections” comprises a proximal end having a cross-sectional diameter of 10 nm-500 nm and a distal end having a cross-sectional diameter of 1 nm-200 nm.

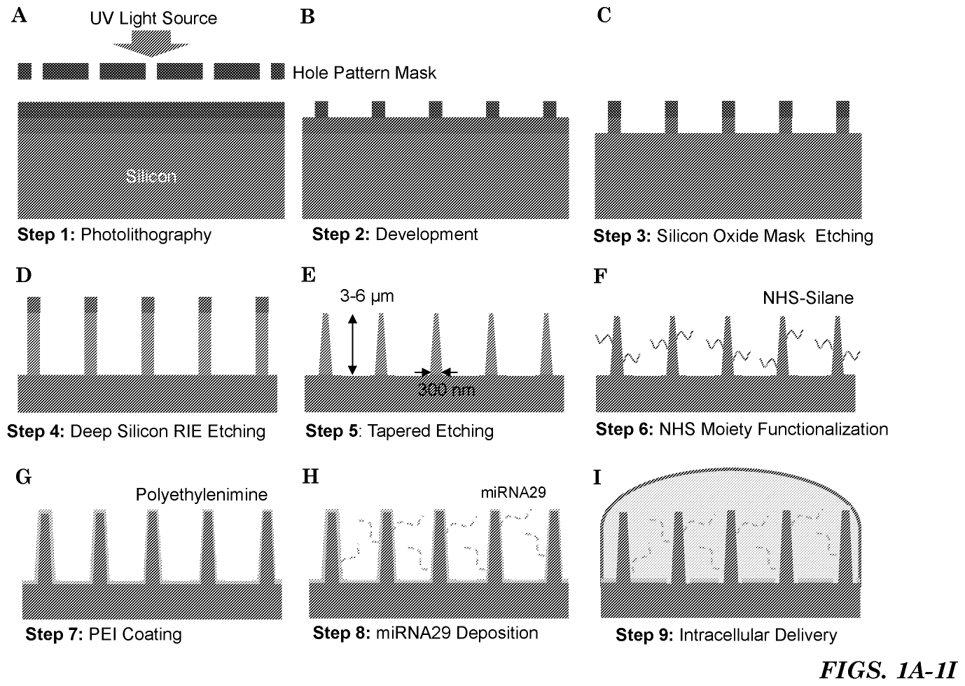

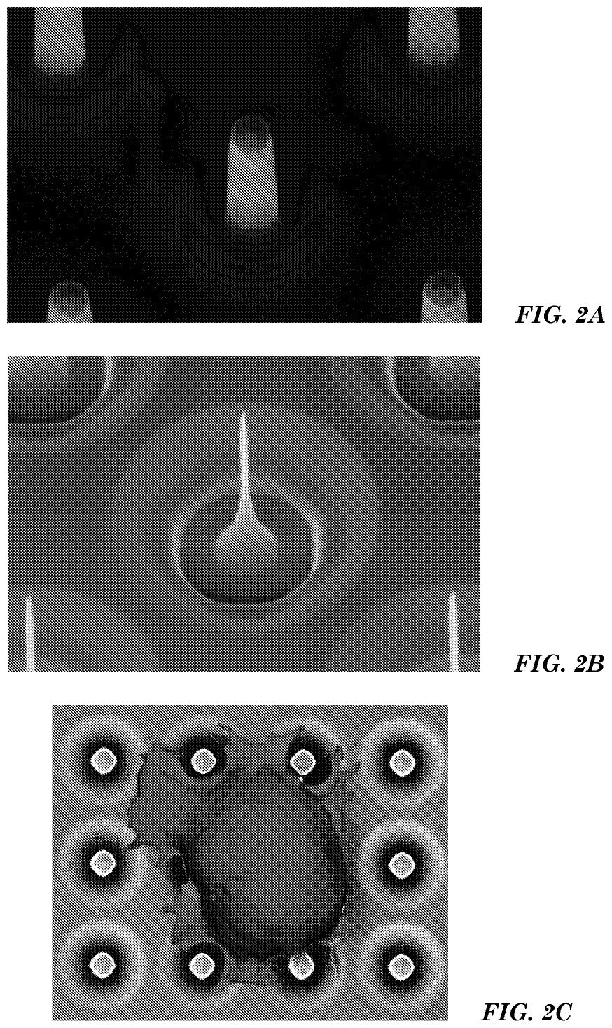

In reference to FIG. 1E and FIG. 2B, the nanoprojection structures comprise a proximal end having a cross-sectional diameter of 300 nm and a distal end having a cross-sectional diameter of ≤10 nm. The tapered nanoprojection structures described herein provide a safety feature which enables the use of the disclosed nanoprojection devices to deliver biomolecules to a target cell while maintaining cell viability. Without being bound by theory, a tapering of the tapered nanoprojection structures provides a narrowed point that can traverse a cell membrane to allow at least a portion of the nanoprojection structure to enter to an interior of the cell and/or can minimize trauma to the cell as the nanoprojection structure enters the cell.

Thus, in some embodiments, the proximal end has a cross-section with a diameter of 10 nm-100 nm, 10 nm-200 nm, 10 nm-300 nm, 10 nm-400 nm, 10 nm-500 nm, 50 nm 100 nm, 50 nm-200 nm, 50 nm-300 nm, 50 nm-400 nm, 50 nm-500 nm, 100 nm-200 nm, 100 nm-300 nm, 100 nm-400 nm, 100 nm-500 nm, 200 nm-300 nm, 200 nm-400 nm, 200 nm-500 nm, 300 nm-400 nm, 300 nm-500 nm, or 400 nm-500 nm. In some embodiments, the proximal end has a cross-section with a diameter of 10 nm-500 nm.

In some embodiments, the distal end has a cross-section with a diameter of 1 nm 10 nm, 1 nm-20 nm, 1 nm-30 nm, 1 nm-40 nm, 1 nm-50 nm, 1 nm-60 nm, 1 nm-70 nm, 1 nm-80 nm, 1 nm-90 nm, 1 nm-100 nm, 1 nm-110 nm, 1 nm-120 nm, 1 nm-130 nm, 1 nm-140 nm, 1 nm-150 nm, 1 nm-160 nm, 1 nm-170 nm, 1 nm-180 nm, 1 nm-190 nm, 10 nm-20 nm, 10 nm-30 nm, 10 nm-40 nm, 10 nm-50 nm, 10 nm-60 nm, 10 nm 70 nm, 10 nm-80 nm, 10 nm-90 nm, 10 nm-100 nm, 10 nm-110 nm, 10 nm-120 nm, 10 nm-130 nm, 10 nm-140 nm, 10 nm-150 nm, 10 nm-160 nm, 10 nm-170 nm, 10 nm-180 nm, 10 nm-190 nm, 20 nm-200 nm, 30 nm-200 nm, 40 nm-200 nm, 50 nm-200 nm, 60 nm-200 nm, 70 nm-200 nm, 80 nm-200 nm, 90 nm-200 nm, 100 nm-200 nm, 110 nm 200 nm, 120 nm-200 nm, 130 nm-200 nm, 140 nm-200 nm, 150 nm-200 nm, 160 nm-200 nm, 170 nm-200 nm, 180 nm-200 nm, or 190 nm-200 nm. In some embodiments, the distal end has a cross-section with a diameter of 100 nm-200 nm.

The nanoprojection structures described herein are solid and at least 0.5 μm-20 μm in length. In various embodiments, the lengths of the nanostructures are in the range of 0.5 μm-5 μm, 0.5 μm-10 μm, 0.5 μm-15 μm, 0.5 μm-20 μm, 1 μm-5 μm, 1 μm-10 μm, 1 μm-15 μm, 1 μm-20 μm, 5 μm-10 μm, 5 μm-15 μm, or 5 μm-20 μm.

In reference to FIG. 1E, the nanoprojection structure may have a length of in the range of 3 μm-6 μm.

The geometry of a nanoprojection structure may be further defined by its “aspect ratio,” which refers to the ratio of the length and the width (or diameter) of the nanoprojection. The one or more nanoprojection structures may be isotropically shaped (i.e., aspect ratio=1) or anisotropically shaped (i.e., aspect ratio 1). Anisotropic nanoprojection structures typically have a longitudinal axis along their length. Exemplary anisotropic nanoprojection structures have aspect ratios of at least 1:2.5, 1:5, 1:10, 1:20, 1:30, 1:40, 1:50, 1:60, 1:70, 1:80, 1:90, 1:100, 1:200, 1:300, 1:400, or 1:500.

FIGS. 1D-1E are schematic diagrams showing a front view of an exemplary nanoprojection device comprising a plurality of nanoprojection structures. In FIG. 1D, the plurality of nanoprojection structures are anisotropically shaped. In FIG. 1E, the plurality of nanoprojection structures are anisotropically shaped.

The silicon nanoprojection device described herein may comprise an array of a plurality of nanoprojection structures. In some embodiments, the one or more nanoprojection structures are spaced 0.5-100 μm apart on the surface of said substrate. For example, the nanoprojection structures may be spaced at least 0.5 μm, 1 μm, 10 μm, 15 μm, 20 μm, 30 μm, 40 μm, 50 μm, 60 μm, 70 μm, 80 μm, 90 μm, or at least 95 μm apart. In some embodiments, the density of the one or more nanoprojections structures on the substrate surface is in the range of 100-400,000 nanoprojection structures/mm². Accordingly, the nanoprojections structures may have a density of 1,000-400,000 nanoprojection structures/mm², 10,000-400,000 nanoprojection structures/mm², 50,000-400,000 nanoprojection structures/mm², 100,000-400,000 nanoprojection structures/mm², 150,000-400,000 nanoprojection structures/mm², 150,000-300,000 nanoprojection structures/mm², or 150,000-200,000 nanoprojection structures/mm².

In reference to FIG. 2C, the density of the one or more nanoprojection structures on the substrate surface is 111,111 nanoprojections/mm².

The nanoprojection device may have an area of at least 1 mm², at least 10 mm², at least 20 mm², at least 30 mm², at least 40 mm², at least 50 mm², at least 60 mm², at least 70 mm², or at least 80 mm².

In some embodiments, the surface of the one or more nanoprojection structures are covalently modified with a modifier. See FIG. 1E. As described herein, the term “covalently modified” refers to the formation of a covalent bond between the one or more nanoprojection structures and the modifier. In some embodiments, the covalent bond is a O—Si bond.

The term “modifier” refers to a compound having a binding group (e.g., a silyl) at one end and a functional group (e.g., N-hydroxysulfosuccinimide (NETS), polyethylene glycol (PEG), 3-(trihydroxy-silyl)-1 propanesulfon, and silane) at the other end.

In some embodiments, the modifier is a silane modifier. As used herein, the term “silane modifier” refers to a compound having a silyl binding group at one end and a functional group (e.g., NETS, sulfonate, or phosphonate) at the other. The silyl binding group forms a covalent bond with the substrate, whereas the functional group is able to interact with ionic compounds. Suitable silane modifiers comprise, e.g., silane-NHS, silane-sulfonate, or silane-phosphonate, octadecyltrichlorosilane, methacrylate silanes, styryl silanes, cyclic azasilanes, vinylsilanes, isocyanate silanes, aminosilanes, glycidoxy silanes, aminopropylmethyldialkoxy-silanes, and mercapto silanes. In some embodiments, the modifier is 3-(trihydroxysilyl)-1-propanesulfonic acid.

The silane modifier may comprises a spacer element (e.g., a polyethylene glycol (PEG) polymer) between the silyl binding group and the functional group (e.g., NHS, sulfonate, or phosphonate). Thus, in some embodiments, the silane modifier is selected from the group consisting of silane-PEG-NHS, silane-PEG-sulfonate, silane-PEG-phosphonate, silane-PEG-biotin, silane-PEG-maleimide, silane-PEG-thiol, silane-PEG-acrylate, silane-PEG-amine, silane-PEG-silane, and silane-PEG-carboxylic acid.

In some embodiments, the modifier is not an amino silane, a glycidoxysilane, and a mercaptosilane. In other embodiments, the modifier is not trimethoxy(octyl)silane, trichloro(propyl)silane, trimethoxyphenylsilane, trimethoxy(2-phenylethyl)silane, allyltriethoxysilane, allyltrimethoxysilane, 3-[bis(2-hydroxyethyl)amino]propyl-triethoxysilane, 3-cyanopropyltriethoxysilane, triethoxy(3-isocyanatopropyl)silane, 3-(trichlorosilyl)propyl methacrylate, and (3-bromopropyl)trimethoxysilane.

In some embodiments, the ionic coating is bonded to or interacting with a modifier, where, the modifier is on the surface of the one or more nanoprojection structures.

As used herein, the term “ionic coating” refers to a coating of an added material, which is different from the modifier. The ionic coating may be a polymer. The term “polymer” refers to a molecule whose structure is composed of multiple repeating units. In some embodiments, the ionic coating is a cationic polymer. Cationic polymers are a class of polymers bearing a positive charge or incorporating cationic entities in their structure. Suitable cationic polymers include, without limitation, polyethyleneimine (PEI), poly-L-lysine (PLL), poly-D-lysine (PDL), poly(diallyldimethylammonium chloride), polyacrylic acid (PAA), polyamideamine epichlorohydrin (PAE), poly(N,N-dimethylaminoethylmethacrylate) (PDMAEMA), and combinations thereof.

PEI is available in a range of sizes and structures, including, without limitation, as linear PEI polymers or branched PEI polymers. In some embodiments, the cationic polymer is a branched PEI having a molecular weight of 25 kDa, 50 kDa, or 270 kDa. In other embodiments, the PEI is a linear PEI having a molecular weight of 22 kDa.

PLL and PDL are positively charged amino acid polymers used as a non-specific attachment factors for cells. When it is absorbed to the nanoprojection structure surface, PLL and/or PDL function to increase the number of positively charged sites available for cell binding. PLL and PDL are available in range of sizes. In some embodiments, the cationic polymer is PLL having a molecular weight of in the range of 30 kDa-70 kDa. In some embodiments, the cationic polymer is PDL having a molecular weight of 100 kDa-300 kDa, 200 kDa-300 kDa, or 100 kDa-200 kDa.

Chitosan is a biocompatible polyelectrolyte, which can form a hydrogel with multivalent anions. In some embodiments, the cationic polymer is chitosan having a molecular weight in the range of 5 kDa-190 kDa or 50 kDa-190 kDa.

Additional suitable ionic coatings include, without limitation, collagen, fibronectin, chitosan, gelatin, dextran, cellulose, cyclodextrin, and laminin.

In some embodiments, the ionic coating comprises an anionic compound. Anionic compounds bearing a negative charge or incorporating anionic entities in their structure. Suitable anionic compounds, without limitation, 3-(trihydroxylsilyl)1-propanesulfon, poly(sodium 4-styrenesulfonate), poly-L-glutamic acid sodium, poly(acrylic acid), and combinations thereof. The anionic compound may be covalently attached to the modifier.

In some embodiments, the silicon nanoprojection device described herein further comprises a biomolecule complexed over and to the ionic coating. See FIG. 1H. In some embodiments, the biomolecule is non-covalently complexed to the ionic coating. For example, the biomolecule may be electrostatically complexed to the ionic coating.

The biomolecule may be selected from the group consisting of a nucleic acid molecule, a protein or peptide fragment, a carbohydrate, a small molecule, and a combination thereof.

As used herein, the term “nucleic acid molecule” refers to a polymeric form of nucleotides of any length, either ribonucleotides or deoxyribonucleotides. Thus, this term includes, but is not limited to, single-, double-, or multi-stranded DNA or RNA, genomic DNA, cDNA, DNA/RNA hybrids, or a polymer comprising purine and pyrimidine bases or other natural, chemically or biochemically modified, non-natural, or derivatized nucleotide bases. In some embodiments, the biomolecule is a nucleic acid molecule selected from the group consisting of an RNA molecule, an DNA molecule, and an aptamer.

Suitable RNA molecules for use in the devices or methods described herein may be selected from the group consisting of a small interfering RNA (siRNA) molecule, a short or small hairpin RNA (shRNA) molecule, a micro RNA (miRNA) molecule, a messenger RNA (mRNA), an antisense oligonucleotide molecule, and a ribozyme.

Small interfering RNA molecules (siRNAs) are double stranded synthetic RNA molecules approximately 20-25 nucleotides in length with short 2-3 nucleotide 3′ overhangs on both ends. The double stranded siRNA molecule represents the sense and anti-sense strand of a portion of a target mRNA molecule. In some embodiments, the siRNA molecules represent the sense and anti-sense of a portion of a mRNA molecule encoding a transcription factor (e.g., T-box protein expressed in T cells (T-BET) or eomesodermin (EOMES)). The sequence of various mRNA molecules encoding transcription factors are readily known in the art and accessible to one of skill in the art for the purposes of designing siRNA oligonucleotides.

siRNA molecules are typically designed to target a region of the mRNA target approximately 50-100 nucleotides downstream from the start codon. Methods and online tools for designing suitable siRNA sequences based on the target mRNA sequences are readily available in the art (see e.g., Reynolds et al., “Rational siRNA Design for RNA Interference,” Nat. Biotech. 2:326-330 (2004); Chalk et al., “Improved and Automated Prediction of Effective siRNA,” Biochem. Biophys. Res. Comm. 319(1): 264-274 (2004); Zhang et al., “Weak Base Pairing in Both Seed and 3′ Regions Reduces RNAi Off-targets and Enhances si/shRNA Designs,” Nucleic Acids Res. 42(19):12169-76 (2014), which are hereby incorporated by reference in their entirety). Upon introduction into a cell, the siRNA complex triggers the endogenous RNA interference (RNAi) pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the application (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety). Methods of constructing DNA-vectors for siRNA expression in mammalian cells are known in the art, see e.g., Sui et al., “A DNA Vector-Based RNAi Technology to Suppress Gene Expression in Mammalian Cells,” Proc. Nat'l Acad. Sci. USA 99(8):5515-5520 (2002), which is hereby incorporated by reference.

Short or small hairpin RNA (shRNA) molecules are similar to siRNA molecules in function, but comprise longer RNA sequences that make a tight hairpin turn. shRNA is cleaved by cellular machinery into siRNA and gene expression is silenced via the cellular RNA interference pathway. Methods and tools for designing suitable shRNA sequences based on the target mRNA sequences (e.g., T-box protein expressed in T cells (T-bet) or eomesodermin (EOMES)) are readily available in the art (see e.g., Taxman et al., “Criteria for Effective Design, Constructions, and Gene Knockdown shRNA Vectors,” BMC Biotech. 6:7 (2006) and Taxman et al., “Short Hairpin RNA (shRNA): Design, Delivery, and Assessment of Gene Knockdown,” Meth. Mol. Biol. 629: 139-156 (2010), which are hereby incorporated by reference in their entirety). Methods of constructing DNA-vectors for shRNA expression and gene silencing in mammalian cells is described herein and are known in the art, see e.g., Cheng et al., “Construction of Simple and Efficient DNA Vector-based Short Hairpin RNA Expression Systems for Specific Gene Silencing in Mammalian Cells,” Methods Mol. Biol. 408:223-41 (2007), which is hereby incorporated by reference in its entirety.

Other suitable RNA molecules for use in the methods described herein include microRNAs (miRNAs). miRNAs are small, regulatory, noncoding RNA molecules that control the expression of their target mRNAs predominantly by binding to the 3′ untranslated region (UTR). A single UTR may have binding sites for many miRNAs or multiple sites for a single miRNA, suggesting a complex post-transcriptional control of gene expression exerted by these regulatory RNAs (Shulka et al., “MicroRNAs: Processing, Maturation, Target Recognition and Regulatory Functions,” Mol. Cell. Pharmacol. 3(3):83-92 (2011), which is hereby incorporated by reference in its entirety). Mature miRNA are initially expressed as primary transcripts known as a pri-miRNAs which are processed, in the cell nucleus, to 70-nucleotide stem-loop structures called pre-miRNAs by the microprocessor complex. The dsRNA portion of the pre-miRNA is bound and cleaved by Dicer to produce a mature 22 bp double-stranded miRNA molecule that can be integrated into the RISC complex; thus, miRNA and siRNA share the same cellular machinery downstream of their initial processing.

microRNAs known to inhibit the expression of transcription factors are well known in the art and suitable for use in the silicon nanoprojection devices or methods described herein. For example, miR-29 is known to modulate the expression of the transcription factors T-bet and EOMES (see, e.g., Steiner et al., “MicroRNA-29 Regulates T-Box Transcription Factors and Interferon-γ Production in Helper T Cells,” Immunity 35(2):169-181 and Kwon et al., “A Systemic Review of miR-29 in Cancer,” Mol. Ther. Oncolytics. 12: 173-194 (2019), which are hereby incorporated by reference in their entirety).

Other suitable RNA molecules for use in the methods described herein include antisense oligonucleotides (ASOs). The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule (e.g., an mRNA molecule encoding a transcription factors T-bet and/or EOMES), to form a double-stranded molecule, which interferes with translation of the mRNA, as the cell will not translate a double-stranded mRNA. Antisense nucleic acids used in the methods of the present application are typically at least 10-15 nucleotides in length, for example, at least 10, 11, 12, 13, 14, 15, 16, 17, 18, 19, 20, 25, 30, 35, 40, 45, 50, 55, 60, 65, 70, 75, or greater than 75 nucleotides in length. The antisense nucleic acid can also be as long as its target nucleic acid with which it is intended to form an inhibitory duplex.

As used herein, the term “ribozyme” refers to a molecule composed of an RNA molecule which functions like an enzyme or a protein including the RNA molecule, and is also called RNA enzyme or catalytic RNA. It has been found that ribozyme is a RNA molecule having a definite tertiary structure, performs a chemical reaction, and has a catalytic or self-catalytic property. Some ribozymes cleave themselves or other RNA molecules to inhibit the activity while other ribozymes catalyze the aminotransferase activity of ribosome. These ribozymes may include hammerhead ribozyme, VS ribozyme, hairpin ribozyme, Group I intron, Group II intron, and the like.

In some embodiments, the biomolecule is a DNA molecule selected from the group consisting of a vector or a plasmid. As used herein, the term “vector” refers to a nucleic acid molecule adapted for transfection into a target cell. Examples of vectors include, but are not limited to, plasmids, cosmids, bacteriophages and the like.

In some embodiments, the biomolecule is a protein selected from the group consisting of a cytokine, a chemokine, a toxin, an antibody, an agonist, an inhibitor, a transcription factor, a protease, an enzyme, and a receptor.

As used herein, the term “cytokine” refers to a protein made by cells that affects the behavior of other cells. Cytokines made by lymphocytes are often called lymphokines or interleukins (ILs). Cytokines act on specific cytokine receptors on the cells that they affect. Exemplary cytokines include, e.g., IFN-α, IFN-β, IFN-γ, B7.1, B7.2, TNF-α, TNF-β, LT-β, CD40L, FasL, CD27L, CD30L, 4-1BBL, Trail, TGF-β, IL-1α, IL-1β, IL-1 RA, IL-10, IL-12, MIF, IL-16, IL-17, and IL-18.

The term “chemokine” refers to a small chemoattractant protein that stimulates the migration and activation of cells, especially phagocytic cells and lymphocytes. Exemplary chemokines include, e.g., IL-8, GROα, GROβ, GROγ, ENA-78, LDGF-PBP, GCP-2, PF4, Mig, IP-10, SDF-1α/β, I-TAC, BLC/BCA-1, MIP-1α, MIP-1β, MDC, TECK, TARC, RANTES, HCC-1, HCC-4, DC-CK1, MIP-3β, MCP-1, MCP-2, MCP-3, MCP-4, Eotaxin, Eotaxin-2/MPIF-2, I-309, MIP-5/HCC-2, 6Ckine, CTACK, MEC, Lymphotactin, and Fractalkine.

The term “toxin” refers to any substance poisonous to an organism. In some embodiments, toxins may be produced by, e.g., bacteria, dinoflagellates, algae, fungi (mycotoxins), higher plants (phytotoxins), and animals (zootoxins). Suitable toxins for use in the device or methods described herein include, without limitation, botulinum toxin.

Suitable antibodies, agonists, inhibitors, and receptors are well known in the art (see, e.g., U.S. Patent Application Publ. No. 2014/0194383, which is hereby incorporated by reference in its entirety).

The term “transcription factor” refers to a protein possessing domains that bind to the DNA of promoter or enhancer regions of specific genes. They also possesses a domain that interacts with RNA polymerase II or other transcription factors and consequently regulate the amount of messenger RNA (mRNA) produced by a gene. Exemplary transcription factors include, e.g., T-bet, Eomes, GATA-1, GATA-2, GATA-3, Ikaros, Ets-1, TCF1, LKLF, NFAT, PU.1, E2a, EBF, SCL, Pax5, Foxp3, STAT1, STAT3, TBP, HER2, AP-2, Nanog, ESR1, TP53, MYC, RELA, POU5F1, SOX2, MAFF, MAFG, MAFK, MITF, ALX4, FOXL2, FOXP2, FOXP3, FOXC1, TAF1, TBX5, LMX1B, STAT3, LXH4, and CTCF.

Suitable enzymes for use in the device or methods described herein include, e.g., kinases; phosphatases; ubiquitin ligases; acetylases; oxo-reductases; lipases; enzymes that add lipid moieties to proteins or remove them; proteases; and enzymes that modify nucleic acids, including but not limited to ligases, helicases, topoisomerases, and telomerases.

In some embodiments, the biomolecule is a small molecule selected from the group consisting of a dye, a quantum dot, and a nanoparticle.

In some embodiments, the biomolecule is a component of or comprises a CRISPR/Cas system. As used herein, the term “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA). An exemplary Cas9 protein is the Streptococcus pyogenes Cas9 protein. Additional Cas9 proteins and homologs thereof are known in the art (see, e.g., Chylinksi, et al., RNA Biol. 10(5):726-737 (2013); Makarova et al., Nat. Rev. Microbiol. 9(6):467-477 (2011); Hou, et al., Proc Natl Acad Sci USA 110(39):15644-9 (2013); Sampson et al., Nature. 497(7448):254-7 (2013); and Jinek, et al., Science. 337(6096):816-21 (2012), which are hereby incorporated by reference in their entirety).

CRISPR/Cas systems may be used to, e.g., edit the genome of a cell. The term “editing” in the context of the present application refers to inducing a structural change in the sequence of the genome at a target genomic region. For example, the editing can take the form of inducing an insertion deletion (indel) mutation into a sequence of the genome at a target genomic region. Such editing can be performed by inducing a double stranded break within a target genomic region, or a pair of single stranded nicks on opposite strands and flanking the target genomic region. Methods for inducing single or double stranded breaks at or within a target genomic region are well known in the art and include the use of a Cas9 nuclease domain, or a derivative thereof, and a guide RNA, or pair of guide RNAs, directed to the target genomic region.

In some embodiments, the silicon nanoprojection device described herein further comprises one or more target cells into which the one or more nanoprojection structures extends. See FIG. 1I.

As described herein, the one or more target cells may be from any organism. For example, the one or more target cells may comprise prokaryotic cells, eukaryotic cells, yeast cells, bacterial cells, plant cells, or animal cells, such as, e.g., reptilian cells, bird cells, fish cells, mammalian cells. In some embodiments, the one or more target cells are animal cells. Accordingly, the one or more target cells may include cells derived from dogs, cats, horses, cattle, sheep, pigs, llamas, gerbils, squirrels, goats, bears, chimpanzees, monkeys, mice, rats, rabbits, etc.

In some embodiments, the animal cells are mammalian cells, e.g., human cells. Suitable cells include primary or immortalized cell lines. As used herein, the term “primary cell” refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some embodiments, the primary cells are adapted to in vitro culture conditions. In some embodiments, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

In some embodiments, the primary cells are hematopoietic cells. As used herein, the term “hematopoietic cell” refers to a cell derived from a hematopoietic stem cell. The hematopoietic cell may be obtained or provided by isolation from an organism, system, organ, or tissue (e.g., blood, or a fraction thereof). Alternatively, a hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, or dendritic cell.

In some embodiments, the one or more target cells is a T cell. Suitable T cells may be selected from the group consisting of inflammatory T cells, cytotoxic T cells, regulatory T cells, helper T cells, or naïve T cells. Representative human T cells are CD34⁺ cells, CD4⁺CD25^{hi}CD127^{lo} regulatory T cells, FOXP3⁺ T cells, CD4⁺CD25^{lo}CD127^{hi} effector T cells, CD4⁺CD8⁺ T cells, CD4⁺ T cells, CD8⁺ T cells, or CD4⁺CD25^{lo}CD127^{hi}CD45RA^{hi}CD45RO⁻ naïve T cells.

As described herein, T cells may be obtained from numerous non-limiting sources, including peripheral blood mononuclear cells, bone marrow, lymph node tissue, umbilical cord, thymus tissue, tissue from an infection site, asthmatic fluid, pleural effusion, spleen tissue, and tumors. In some embodiments, the one or more T cells may be derived from a healthy donor, a subject who has been diagnosed with cancer, or a subject who has been diagnosed with an infection. In other embodiments, the one or more T cells is part of a mixed population of cells having different phenotypic characteristics. Also within the scope of the present application is a line of cells obtained according to the methods described herein above.

Additional exemplary cell types for use in the methods described herein include, without limitation, placental cells, keratinocytes, basal epidermal cells, urinary epithelial cells, salivary gland cells, mucous cells, serous cells, von Ebner's gland cells, mammary gland cells, lacrimal gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing α cells, somatostatin-producing δ cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair cells, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian cells, Sertoli cells, and thymus epithelial cells.

The one or more target cells for use in the methods of the present application include fetal cells, or adult cells, at any stage of their lineage, e.g., pluripotent, multipotent, or differentiated cells.

In some embodiments, the one or more target cells comprise pluripotent stem cells. Pluripotent stem cells can give rise to any cell of the three germ layers (i.e., endoderm, mesoderm and ectoderm). In one embodiment, the one or more target cells comprise induced pluripotent stem cells (iPSCs). In another embodiment, the one or more target cells comprise pluripotent embryonic stem cells.

In another embodiment, the one or more target cells comprise multipotent stem cells. Multipotent stem cells can develop into a limited number of cells in a particular lineage. Examples of multipotent stem cells include progenitor cells. Progenitor cells are an immature or undifferentiated cell population having the potential to mature and differentiate into a more specialized, differentiated cell type. A progenitor cell can also proliferate to make more progenitor cells that are similarly immature or undifferentiated. Suitable progenitor cells for use in the methods disclosed herein include, without limitation, bone marrow progenitor cells, cardiac progenitor cells, endothelial progenitor cells, epithelial progenitor cells, hematopoietic progenitor cells, hepatic progenitor cells, osteoprogenitor cells, muscle progenitor cells, pancreatic progenitor cells, pulmonary progenitor cells, renal progenitor cells, vascular progenitor cells, retinal progenitor cells, neural progenitor cells, neuronal progenitor cells, and glial progenitor cells.

The one or more target cells may comprise terminally differentiated cells. In some embodiments, the one or more target cells comprise terminally differentiated adipocytes, chondrocytes, endothelial cells, epithelial cells (keratinocytes, melanocytes), bone cells (osteoblasts, osteoclasts), liver cells (cholangiocytes, hepatocytes), muscle cells (cardiomyocytes, skeletal muscle cells, smooth muscle cells), retinal cells (ganglion cells, muller cells, photoreceptor cells), retinal pigment epithelial cells, renal cells (podocytes, proximal tubule cells, collecting duct cells, distal tubule cells), adrenal cells (cortical adrenal cells, medullary adrenal cells), pancreatic cells (alpha cells, beta cells, delta cells, epsilon cells, pancreatic polypeptide producing cells, exocrine cells); lung cells, bone marrow cells (early B-cell development, early T-cell development, macrophages, monocytes), urothelial cells, fibroblasts, parathyroid cells, thyroid cells, hypothalamic cells, pituitary cells, salivary gland cells, ovarian cells, testicular cells, neurons, oligodendrocytes, or astrocytes.

In some embodiments, the one or more target cells comprise transgenic cells from cultures or from transgenic organisms. The cells may be from a specific tissue, body fluid, organ (e.g., brain tissue, nervous tissue, muscle tissue, retina tissue, kidney tissue, liver tissue, etc.), or any derivative fraction thereof. The term includes healthy cells, transgenic cells, cells affected by internal or exterior stimuli, cells suffering from a disease state or a disorder, cells undergoing transition (e.g., mitosis, meiosis, apoptosis, etc.), etc.

In some embodiments, the one or more target cells are bacterial cells. Suitable bacterial cells include, e.g., Agrobacterium (e.g., Agrobacterium tumefaciens); Bacillus (e.g., Bacillus cereus, Bacillus subtilis, Bacillus thuringiensis, Bacillus weihenstephanensis); Bartonella (e.g., Bartonella henselae, Bartonella schoenbuchensis); Bdellovibrio (e.g., Bdellovibrio bacteriovorus, Bdellovibrio starri, Bdellovibrio stolpii); Bifidobacterium (e.g., Bifidobacterium adolescentis, Bifidobacterium bifidum, Bifidobacterium lactis, Bifidobacterium longum); Bordetella (e.g., Bordetella pertussis); Borrelia (e.g., Borrelia burgdorferi); Brucella (e.g., Brucella abortus, Brucella bronchiseptica); Burkholderia (e.g., Burkholderia cenocepacia, Burkholderia fungorum, Burkholderia mallei, Burkholderia pseudomallei); Campylobacter (e.g., Campylobacter fecalis, Campylobacter pylori, Campylobacter sputorum); Chlamydia (e.g., Chlamydia pneumoniae, Chlamydia psittaci, Chlamydia trachomatis); Clostridium (e.g., Clostridium difficile, Clostridium novyi, Clostridium oncolyticum, Clostridium perfringens, Clostridium sporogenes, Clostridium tetani); Corynebacterium (e.g., Corynebacterium diphtheriae, Corynebacterium glutamicum, Corynebacterium jeikeium); Edwardsiella (e.g., Edwardsiella hoshinae, Edwardsiella ictaluri, Edwardsiella tarda); Enterobacter (e.g., Enterobacter aerogenes, Enterobacter cloacae, Enterobacter sakazakii); Enterococcus (e.g., Enterococcus avium, Enterococcus faecalis, Enterococcus faecium, Enterococcus gallinarum); Escherichia (e.g., Escherichia coli); Eubacterium (e.g., Eubacterium lentum, Eubacterium nodatum, Eubacterium timidum); Helicobacter (e.g., Helicobacter pylori); Klebsiella (e.g., Klebsiella oxytoca, Klebsiella pneumoniae); Lactobacillus (e.g., Lactobacillus bulgaricus, Lactobacillus casei, Lactobacillus delbrueckii, Lactobacillus plantarum); Lactobacterium (e.g., Lactobacterium fermentum); Lactococcus (e.g., Lactococcus lactis, Lactococcus plantarum); Legionella (e.g., Legionella pneumophila); Listeria (e.g., Listeria innocua, Listeria ivanovii, Listeria monocytogenes); Microbacterium (e.g., Microbacterium arborescens, Microbacterium lacticum); Mycobacterium (e.g., Bacille Calmette-Guerin (BCG), Mycobacterium avium, Mycobacterium bovis, Mycobacterium paratuberculosis, Mycobacterium tuberculosis); Neisseria (e.g., Neisseria gonorrhoeae, Neisseria lactamica, Neisseria meningitidis; Pasteurella (e.g., Pasteurella haemolytica, Pasteurella multocida); Salmonella (e.g., Salmonella bongori, Salmonella enterica ssp.; Shigella (e.g., Shigella dysenteriae, Shigella flexneri, Shigella sonnei); Staphylococcus (e.g., Staphylococcus aureus, Staphylococcus lactis, Staphylococcus saprophyticus; Streptococcus (e.g., Streptococcus gordonii, Streptococcus lactis, Streptococcus pneumoniae, Streptococcus pyogenes, Streptococcus salivarius); Treponema (e.g., Treponema denticola, Treponema pallidum); Vibrio (e.g., Vibrio cholerae); Yersinia (e.g., Yersinia enterocolitica, Yersinia pseudotuberculosis).

In some embodiments, the one or more target cells are plant cells. As used herein, the term “protoplast” refers to a plant cell that has had its protective cell wall partly or totally removed, e.g., by enzymatic treatment resulting in an intact biochemical competent unit of living plant that can regenerate the cell wall and further grow into a whole plant under proper growing conditions. Plant protoplasts may be derived from plant leaves, roots, shoot apices, fruits, embryos, and microspores. In some embodiments, the plant cell or plant protoplast is derived from, e.g., Solanum lycopersicon, Nicotiana tabaccum, Brassica napus, Daucus carota, Lactucca sativa, Zea mays, Nicotiana benthamiana, Petunia hybrida, Solanum tuberosum, or Oryza sativa.

The various types of cells that are used herein are grown and cultured according to methods well known in the art. Generally, a cell culture medium contains a buffer, salts, energy source, amino acids (e.g., natural amino acids, non-natural amino acids, etc.), vitamins, and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites.

Another aspect of the present application relates to a pair of silicon nanoprojection devices between the substrates of which the one or more target cells are sandwiched.

Yet another aspect of the present application relates to a method of making a nanoprojection device. This method involves providing a silicon monolithic structure and carrying out a series of nanofabrication steps on the silicon monolithic structure to form one or more nanoprojection structures having a proximal end attached to a surface of a substrate and extending away from the surface of the substrate to a distal end. The one or more nanoprojection structures have a configuration which tapers narrowingly from the proximal end to the distal end.

The nanoprojection devices according to the present application may be obtained using a “top-down” fabrication process that involves removing predefined structures from the silicon monolithic structure. For example, the sites where the one or more nanoprojection structures are to be formed may be patterned into a resist layer and subsequently etched to develop the patterned sites into three-dimensional nanoprojection structures.

In some embodiments, the nanofabrication steps involves: depositing an etching mask layer onto the silicon monolithic structure; coating the deposited etching mask layer with resist layer; patterning the silicon monolithic structure with the resist coated mask layer, using lithography, to produce, upon development, one or more nanoprojection structures extending from the surface; developing the patterned silicon monolithic structure with the coated mask layer into one or more nanoprojection structures extending from the surface using mask etching and deep silicon reactive-ion etching (RIE); and tapering the nanoprojection structures using tapered etching.

The etching mask layer may be a silicon dioxide layer, a polymer layer, or a metal layer. In some embodiments, the etching mask layer is selected from the group consisting of silicon oxide, silicon dioxide, silicon nitride, silicon carbide, iron oxide, aluminum oxide, iridium oxide, tungsten, stainless steel, silver, platinum, gold, aluminum, copper, molybdenum, tantalum, titanium, nickel, chromium, and palladium.

In some embodiments, the etching mask layer has a thickness in the range of 1,000 Å-5,000,000 Å (100 nm-500 μm); 1,000 Å-1,000,000 Å (100 nm-100 μm); 1,000 Å-100,000 Å (100 nm-10 μm); 1,000 Å-10,000 Å (100 nm-1 μm); 2,000 Å-5,000 Å (200 nm-500 nm); 3,000 Å-5,000 Å (300 nm-500 nm); or 4000 Å-5000 Å (400 nm-500 nm). In some embodiments, the etching mask layer is approximately 3,000 Å (300 nm) thick. For example, the etching mask layer may be a silicon dioxide layer having a thickness of approximately 3,000 Å (300 nm).

Methods of depositing etching mask layers are well known in the art and include, e.g., wet oxide annealing, dry oxide annealing, and chemical vapor deposition (CVD). As used herein, “dry oxide annealing” refers to a process in which a silicon substrate is placed in a pure oxygen gas (O₂) environment and the silicon atoms on the surface of the substrate react with the oxide gas to produce a silicon oxide film of approximately 1000 Å (100 nm). As used herein, “wet oxide annealing” refers to a process in which a silicon substrate is placed into an atmosphere of water vapor (H₂O) and the silicon atoms on the surface of the substrate react with the water vapor molecules to produce a silicon oxide film of approximately 1000 Å-5000 Å (100 nm-500 nm). As used herein, “chemical vapor deposition” refers to process in which films of materials are deposited from the vapor phase by means of a chemical reaction between volatile precursors and the surface of the materials to be coated. As the precursor gases pass over the surface of the heated substrate, the resulting chemical reaction forms a solid phase which is deposited onto the substrate. CVD processes are well known in the art and include, e.g., atmospheric pressure chemical vapor deposition, metal-organic chemical vapor deposition, low pressure chemical vapor deposition, laser chemical vapor deposition, photochemical vapor deposition, chemical vapor infiltration, chemical beam epitaxy, plasma-assisted chemical vapor deposition and plasma-enhanced chemical vapor deposition (see, e.g., A. S. H. Makhlouf, “Current and Advanced Coating Technologies for Industrial Applications,” in Nanocoatings and Ultra-Thin Films pp. 3-23 (2011), which is hereby incorporated by reference in its entirety).

In some embodiments, the deposited etching mask layer is coated with a positive resist layer. As used herein, the term “positive resist” refers to a material that becomes soluble to a resist developer after being exposed to a beam of photons or electrons. When a beam of photons is used, the technique is generally termed photolithography, and when a beam of electrons is used, the technique is generally referred to as electron beam lithography. Examples of positive resists used in photolithography include, but are not limited to, poly(methyl methacrylate) (PMMA) and SPR220, S1800, and ma-P1200 series photoresists. Other examples of photoresists include, but are not limited to, SU-8, S1805, LOR 3A, poly(methyl glutarimide), phenol formaldehyde resin (diazonaphthoquinone/novolac), diazonaphthoquinone (DNQ), Hoechst AZ 4620, Hoechst AZ 4562, Shipley 1400-17, Shipley 1400-27, Shipley 1400-37, or the like. Examples of positive resists used in electron beam lithography include, but are not limited to, PMMA, ZEP 520, APEX-E, EBR-9, and UVS. In some embodiments, portions of the resist may be exposed to light (visible, UV, etc.), electrons, ions, X-rays, etc. (e.g., projected onto the photoresist), and the exposed portions can be etched away (e.g., using suitable etchants, plasma, etc.) to produce a suitable pattern.

In some embodiments, the deposited etching mask layer is coated with a negative resist layer. As used herein, the term “negative resist” refers to a material that becomes less soluble to a resist developer after being exposed to a beam of photons or electrons. Several non-limiting examples of negative resists used in photolithography include SU-8 series photoresists, KMPR 1000, and UVN30. Additional non-limiting examples of negative resists used in electron beam lithography include hydrogen silsesquioxane (HSQ) and NEB-31.

In carrying the methods of the present application, it should be appreciated that any positive resist, negative resist, or resist developer known in the art may be used. Resist developers for photolithography include aqueous solutions with either an organic compound such as tetramethylammonium hydroxide or an inorganic salt such as potassium hydroxide, and they may also contain surfactants. Resist developers for electron beam lithography may include methyl isobutyl ketone and isopropyl alcohol.

Reference is now made to FIGS. 1A-1E, which provide a schematic representation of the nanofabrication steps involved in fabricating an exemplary nanoprojection device of the present application. In FIG. 1A, a silicon monolithic structure is deposited with an etching mask layer (e.g., a silicon dioxide (SiO₂) layer) and the etching mask layer is then coated with a negative photoresist layer. Next, deep UV photolithography is used to pattern the negative photoresist layer. The patterned negative photoresist layer is developed using AZ® 726 MIF (available from MicroChemicals, Ulm, Germany). In FIG. 1B, fine patterns of nanoprojection arrays were developed to produce nanostructures in the negative photoresist layer.

Mask etching may be carried out using wet etching, dry etching, or combinations of wet and dry etching. Suitable wet and dry etching techniques are well known in the art. As described herein, developing the patterned silicon monolithic structure may be carried out by silicon oxide mask etching. Silicon oxide mask etching may involve plasma etching and/or reactive ion etching (RIE). Thus, developing the patterned silicon monolithic structure may be carried out to remove portions of the etching mask layer. By varying the developing conditions (etching processes, rates, times), it is possible to manipulate the amount of the etching mask layer that is removed and thereby manipulate the dimensions of the one or more nanoprojection structures.

In some embodiments, dry plasma etching is carried out by exciting molecules of a gas to form reactive ions, and exposing the surface to be etched to these reactive ions. The reactive ions then eat into the exposed surface, removing surface to produce one or more structures in the exposed surface. In some embodiments, dry plasma etching is carried out using a fluorocarbon gas (e.g., CHF₃) or a combination of a fluorocarbon gas and H₂ or O₂. For example, dry plasma etching may be carried out using a combination of CHF₃ and O₂. Dry plasma etching may be carried out using a combination of CHF₃ and O₂ to achieve an etching rate in the range of 100 nm/minute-200 nm/minute. In some embodiments, the etching rate is approximately 150 nm/minute. In some embodiments, plasma etching is carried out for 1 minute-10 minutes, 1 minute-5 minutes, or 1 minute-3 minutes. In some embodiments, plasma etching is carried out for at least 1 minute, at least 2 minutes, or at least 3 minutes.

In FIG. 1C, the pattern of the nanostructures in the photoresist layer is transferred to the SiO₂ layer using dry etching.

The term “reactive ion etching” refers to a process by which plasma in reaction is formed by a high frequency electric field applied between two fixed electrodes. The electric field defines the direction of plasma movement, allowing for the formation of anisotropic nanoprojection structures. RIE etching may be carried out using a halogen gas (e.g., HF, HCl, HBr, F₂, Cl₂, Br₂) alone or in combination with an inert gas (e.g., He, Ar, or N₂). In some embodiments, RIE etching is carried out using a combination of HBr and Ar (see, e.g., U.S. Pat. No. 5,007,982, which is hereby incorporated by reference in its entirety). The RIE etching process may be carried out at a rate of 100-200 nm/minute. In some embodiments, the etching rate is approximately 156 nm/minute. In some embodiments, RIE etching is carried out for 1 minute-30 minutes, 5 minutes-25 minutes, 10 minutes-20 minutes, or 15 minutes-18 minutes. In one embodiment, the RIE etching process is carried out for 18 minutes.

In FIG. 1D, deep silicon RIE etching is carried out to produce isotropically shaped nanostructures. Images of exemplary isotropically shaped nanostructures depicted in FIG. 1D are shown in FIG. 2A.

As described herein, the length of the nanoprojection structures can vary with the etching time and thickness of the deposited etching mask layer (e.g., the SiO₂ layer). Exemplary nanoprojection structure lengths are identified in more detail above.

Tapering the nanoprojection structures using tapered etching may be carried out using a fluorocarbon gas (e.g., CHF₄). The CHF₄ etching may be carried out at a rate of 10 nm-100 nm/minute, 20 nm-100 nm/minute, 30 nm-100 nm/minute, 40 nm-100 nm/minute, 50 nm-100 nm/minute, 60 nm-100 nm/minute, 80 nm-100 nm/minute, or 90 nm-100 nm/minute. In some embodiments, the tapered etching process may be carried out for 71 nm/minute. The amount of time tapered etching is carried out depends on the diameter of the distal end. In some embodiments, the tapered etching process is carried out for at least 2 minutes, at least 3 minutes, at least 4 minutes, at least 4 minutes, at least 10 minutes, at least 20 minutes, at least 25 minutes, or at least 30 minutes, or more. In some embodiments, the tapered etching process is carried out for 1 minute-30 minutes. In other embodiments, the tapered etching process is carried out for 22 minutes.

In FIG. 1E, tapered etching is carried to produce anisotropically shaped nanoprojection structures.

In some embodiments, the method further involves covalently modifying a surface of the one or more nanoprojection structures with a modifier (e.g., silane-PEG-NHS or 3-(trihydroxysilyl)-1-propanesulfonic acid). Additional suitable modifiers are described in detail above. In some embodiments, modifying the one or more nanoprojection structures is carried out by covalently modifying the surface of the nanoprojection structure.

In some embodiments, the method further involves conjugating a polymer (e.g., a cationic or anionic polymer) to the modified one or more nanoprojection structures. Suitable polymers include PEI, PLL, chitosan, and combinations thereof. Additional suitable polymers are described in detail above.

Polymers may be deposited by, e.g., spin coating. In one embodiment, PEI is deposited onto the surface of a modified nanoprojection device (e.g., a silane-PEG-NHS modified device) by spin coating.

In some embodiments, an anionic coating (e.g., 3-(trihydroxysilyl)-1-propanesulfon) is deposited onto a modified nanoprojection device by vapor phage coating.

In some embodiments, the method further involves complexing biomolecule on the one or more modified, polymer coated nanoprojection structures.

In some embodiments, the biomolecule is selected from the group consisting of a nucleic acid molecule, a protein or peptide fragment, a carbohydrate, a small molecule, and a combination thereof. Additional suitable biomolecules are described in more detail above.

A further aspect of the present application relates to a method for delivering a biomolecule to a target cell. This method involves providing a silicon nanoprojection device according to the present application and contacting one or more target cells with the one or more nanoprojection structures of the silicon nanoprojection device, so that the one or more nanoprojection structures extend into the one or more target cells.

As described herein above, the one or more target cells may comprise prokaryotic cells, eukaryotic cells, yeast cells, bacterial cells, plant cells, and/or animal cells. In some embodiments, the animal cells are mammalian cells, e.g., human cells. Suitable cells for use in the methods described herein include primary or immortalized cells, fetal cells, or adult cells, at any stage of their lineage, e.g., pluripotent, multipotent, or differentiated cells.

The one or more target cells for use in the methods described herein may be selected from a group consisting of a normal cell, benign cell, cancer cell, immortalized cell, genetically engineered cell, stem cell, and a patient derived cells, or a combination thereof.

In some embodiments, the one or more target cells are bacterial cells. Suitable bacterial cells are described in detail above.

In other embodiments, the one or more target cells is a plant cell or a plant protoplast. Suitable plant cell and plant protoplasts for use in the methods of the present application are described in more detail above.

Suitable biomolecules for delivery are described in detail above.

In some embodiments, the method further involves centrifuging the silicon nanoprojection device during said contacting to deliver the biomolecule into the target cell. Centrifuging may be carried out at 500-100×g for 1-10 minutes.

In some embodiments, the method further involves providing a second one of the silicon nanoprojection devices having one or more nanoprojection structures complexed with a biomolecule and contacting the one or more target cells with the second one of the silicon nanoprojection device to form a sandwich structure of the one or more target cells between the first and the second silicon nanoprojection devices.

Additional aspects relates to one or more target cells produced according to the methods described herein. In some embodiments, the one or more target cells comprise one or more biomolecules. Suitable biomolecules are described in detail above. For example, the one or more target cells may comprise a heterologous biomolecule selected from the group consisting of a nucleic acid molecule, a protein or peptide fragment, a carbohydrate, a small molecule, or a combination thereof.

Another aspect of the present application relates to a method of treating a subject with a modified cell, the method comprising selecting a subject in need of treatment with a modified cell and administering one or more modified target cells as described herein to treat the selected subject.

As used herein, a “subject” or a “patient” suitable for administering the one or more target cell according to the present application encompasses any animal. For example, the animal may be a mammal. Suitable subjects include, without limitation, domesticated and undomesticated animals such as dogs, cats, horses, cattle, sheep, pigs, llamas, gerbils, squirrels, goats, bears, chimpanzees, monkeys, mice, rats, rabbits, etc. In one embodiment the subject is a human subject. Suitable human subjects include, without limitation, infants, children, adults, and elderly subjects.

In some embodiments, the subject is suffering from a disease or disorder. The term “disease” or “disorder” includes metabolic diseases (e.g., obesity, cachexia, diabetes, anorexia, etc.), cardiovascular diseases (e.g., atherosclerosis, ischemia/reperfusion, hypertension, restenosis, arterial inflammation, etc.), immunological disorders (e.g., chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy, etc.), nervous system disorders (e.g., neuropathies, Alzheimer disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.), oncological disorders (e.g., cancer).

In some embodiments, the disease or disorder is cancer. As used herein, the term “cancer” refers to or describes the physiological condition in which a population of cells are characterized by abnormal, unrestrained growth with the potential to cause detrimental local mass effects, or to spread to other parts of the body through the lymphatic system or bloodstream.

Examples of cancer include, but are not limited to, carcinoma, sarcoma, melanoma, leukemia, lymphoma, and combinations thereof (mixed-type cancer). A “carcinoma” is a cancer originating from epithelial cells of the skin or the lining of the internal organs. A “sarcoma” is a tumor derived from mesenchymal cells, usually those constituting various connective tissue cell types, including fibroblasts, osteoblasts, endothelial cell precursors, and chondrocytes. A “melanoma” is a tumor arising from melanocytes, the pigmented cells of the skin and iris. A “leukemia” is a malignancy of any of a variety of hematopoietic stem cell types, including the lineages leading to lymphocytes and granulocytes, in which the tumor cells are nonpigmented and dispersed throughout the circulation. A “lymphoma” is a solid tumor of the lymphoid cells. More particular examples of such cancers include, e.g., acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), mixed-cell carcinoma, mucinous (colloid) carcinoma, mucinous cystadenocarcinoma, papillary adenocarcinoma, pleomorphic giant-cell carcinoma, serous cystadenocarcinoma, and small-cell (oat-cell) carcinoma.

The cancer may be selected from the group consisting of adrenocortical cancer, anal cancer, astrocytoma, basal cell carcinoma, bile duct cancer, bladder cancer, bone cancer, glioma, breast cancer, bronchial adenomas/carcinoids, cervical cancer, colon cancer, colorectal cancer, endometrial cancer, ependymoma, esophageal cancer, eye cancer, glioma, head and neck cancer, squamous cell head and neck cancer, hepatocellular cancer, hypopharyngeal cancer, islet cell carcinoma, Kaposi's sarcoma, laryngeal cancer, liver cancer, lung cancer, melanoma, Merkel cell carcinoma, mesothelioma, nasopharyngeal cancer, neuroblastoma, oral cancer, osteosarcoma, ovarian cancer, pancreatic cancer, parathyroid cancer, penile cancer, pharyngeal cancer, prostate cancer, rectal cancer, retinoblastoma, rhabdomyosarcoma, oral cavity cancer, gastrointestinal cancer, small intestine cancer, testicular cancer, throat cancer, thyroid cancer, urethral cancer, and uterine cancer.

In some embodiments, the cancer is a hematological cancer. These cancers, also known as blood cancers, are a group of diverse cancers originated from bone marrow or lymphatic tissues, affecting blood functions. Hematological cancers include, for example, lymphoma, leukemia, myeloma or a lymphoid cancer, as well as a cancer of the spleen and the lymph nodes. Exemplary lymphomas include both B cell lymphomas and T cell lymphomas. Non-limiting examples of B cell lymphomas include diffuse large B cell lymphoma (DLBCL), follicular lymphoma (FL), mucosa-associated lymphatic tissue lymphoma (MALT), small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma.

In some embodiments, the one or more target cells is a primary cell (e.g., a primary human cell, a primary rodent cell, or a primary feline cell). In other embodiments, the one or more target cells is a cell line derived from a primary cell.

Suitable target cells are described in detail above and include, e.g., lymphocytes (T lymphocytes or B lymphocytes).

In some embodiments, the subject is a plant. The plant may be selected from, e.g., Solanum lycopersicon, Nicotiana tabaccum, Brassica napus, Daucus carota, Lactucca sativa, Zea mays, Nicotiana benthamiana, Petunia hybrida, Solanum tuberosum, or Oryza sativa.

In carrying out the methods of the present application, “treating” or “treatment” includes inhibiting, preventing, ameliorating or delaying onset of a particular disease or disorder. Treating and treatment also encompasses any improvement in one or more symptoms of the disease or disorder. Treating and treatment encompasses any modification to the disease condition or course of disease progression as compared to the disease condition in the absence of therapeutic intervention.

In some embodiments, the administering is effective to reduce at least one symptom of a disease or disorder that is associated with the target cell type. In another embodiment, the administering is effective to mediate an improvement in the disease or disorder that is associated with the loss or dysfunction of the target cell type. In another embodiment, the administering is effective to prolong survival in the subject as compared to expected survival if no administering were carried out.

In accordance with this aspect of the present application, the one or more target cells may be autologous/autogeneic (“self”) to the recipient subject. In another embodiment, the one or more target cells is non-autologous (“non-self,” e.g., allogeneic, syngeneic, or xenogeneic) to the recipient subject.

In some embodiments, the one or more target cells is administered to a subject in one dose. In others, the one or more target cells is administered to a subject in a series of two or more doses in succession. In some other embodiments where the one or more target cells is administered in a single dose, in two doses, and/or more than two doses, the doses may be the same or different, and they are administered with equal or with unequal intervals between them.

The one or more target cells may be administered in many frequencies over a wide range of times. In some embodiments, they are administered over a period of less than one day. In other embodiments, they are administered over two, three, four, five, or six days. In some embodiments, they are administered one or more times per week, over a period of weeks. In other embodiments, they are administered over a period of weeks for one to several months. In various embodiments, they may be administered over a period of months. In others they may be administered over a period of one or more years. Generally, lengths of treatment will be proportional to the length of the disease process, the effectiveness of the therapies being applied, and the condition and response of the subject being treated.

The choice of formulation for administering the one or more target cells for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Thus, measures may be taken to increase cell survival and/or to overcome problems posed by barriers to seeding and/or growth.

Final formulations may include an aqueous suspension of cells/medium and, optionally, protein and/or small molecules, and will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose, and the like. Organic polymer base materials, such as polyethylene glycol and hyaluronic acid as well as non-fibrillar collagen, such as succinylated collagen, can also act as lubricants. Such lubricants are generally used to improve the injectability, intrudability, and dispersion of the injected material at the site of injection and to decrease the amount of spiking by modifying the viscosity of the compositions. This final formulation is by definition the cells described herein in a pharmaceutically acceptable carrier.

### EXAMPLES

The examples below are intended to exemplify the practice of embodiments of the application but are by no means intended to limit the scope thereof.

### Materials and Methods for Examples 1-9

Murine CD8⁺ T Cell Enrichment from Spleen. Murine CD8⁺ T cells were isolated from B6×gB homo mice (n=3) by removing spleens. Isolated spleens were placed in 3 ml RP-10 medium and mashed through a 40 μm screen. Cells were then washed twice with 5 ml RP-10 and pelleted by centrifugation (500×g for 5 minutes). Washed cells were resuspended in 75 μl CD8a (Ly-2) MicroBeads (Miltenyi Biotech)+675 μl MACS®/tube per spleen. Resuspended cells were incubated for 10 minutes at 4° C. and washed twice with MACS®.

Complexing PEI-Coated Nanoprojection Arrays with mir-29, mir-130, or control miRNA. PEI-coated nanoprojection arrays were complexed with mir-29, mir-130, or control miRNA by adding either 70 ul (20 uM) mir-29 FITC+mir-130 APC (1:1) or inert-FITC+inert-APC to PEI-coated nanoprojection arrays. The arrays were left undisturbed in the dark to dry for ˜4 hrs in a 24 well plate.

Feline Cell Culture. 65 μl of feline CD8⁺ T cells (2M cells total) were added to the surface of dried or partially dried miRNA complexed PEI-coated Nanoprojection Arrays (at a density of 2 million cells/device). Arrays were incubated for 20 minutes undisturbed. Next, each array was carefully centrifuged at 200×g for 5 minutes (4 acceleration; 5 deceleration). Each well was filled with 300 μl PR-10+hIL-2 (20 ng/ml) and incubated at 37° C. for 4 days.

Naïve Phenotyping. 50 μl of cells were transferred to 96 round well plates and washed with 1× with 150 μl FACS buffer and 1× with 200 μl FACS buffer. Cells were then stained with PANEL 1 (Table 1) surface antibodies in FACS buffer for 30 minutes. Cells were washed 1× with 200 μl FACS buffer. 250 μl of cold Fix/Perm solution was added to each well. Next, 1 ml of FIX/PERM solution was combined with 3 ml of diluent. Cells were pipetted up and down several times to mix and incubated at 4° C. for 45 minutes. Cells were then pelleted by centrifugation and washed 2× with Invitrogen™ eBioscience™ Permeabilization Buffer 1× Perm Wash. Next, cells were incubated with PANEL 1 (Table 1) intracellular antibodies in 1× Perm Wash for 30 minutes, followed by washing 2× with 1× Perm Wash solution.

miRNA+Target QPCR. After 4 days incubation with nanoprojection arrays, cells were harvested and washed 2× with RP-10. Cells were pelleted by centrifuging at 600 g for 4 minutes.

Post Nanoprojection—TCR Stim (0 hour, 24 hours, 48 hours, 72 hours proliferation). T cells incubated on nanoprojection arrays were labeled with CFSE as follows. CFSE dye stock was diluted 1:500 in a sterile room with room temperature PBS=10 μM. Cells were mixed with e450 Proliferation Dye working solution at 1:1 ratio and incubated for 5 minutes in the dark at room temperature. 5-10× room temperature FBS was added to quench staining. Cells were next washed 1× with RP-10, resuspended at 2×10⁶ cells/ml in RP-10+1L-2 (2 ng/ml), and plated at 1000 μl per well in 96 round well plate. 100 μl of IL-2 media was added (these are at 2× concentration): IL-2+gB peptide (10-9M). At the 0 hour and 72 hour time points, 175 μl MACS® was added to cells. Cells were pelleted, resuspended in 200 μl FACS, pelleted, incubated with 50 μl of PANEL 2 surface antibody cocktail per plate, stained for 30 minutes at 4° C. in the dark, washed with 200 μl FACS, resuspended in 50 μl IC fixation buffer, incubated for 20 minutes, and washed with 200 μl FACS.

Post Nanoprojection Array—24 hours GB Peptode Stim+BFA (Cytokine). Nanoprojection structure incubated cells were aliquoted at 100 μl/well in a 96 round well plate. Next, 100 μl/well of 2× peptide in RP10 was added at 10-7M. Cells were pipetted up/down and cells were incubated, in the plate, undisturbed, at 37° C. for 24 hours. The next morning, at 3 μl/well of BFA was added and cells were incubated at 37° C. for 5 hours. Cells were pelleted, washed 1× with FACS, resuspended in PANEL 3 (Table 1) surface stain (50 μl) made in FACS, and incubated for 30 minutes at 4° C. Next, cells were washed 2× in FACS buffer, resuspended in 50 μl IC Fixation buffer, incubated at 4° C. for 10 minutes, diluted with 150 μl 1× Perm, incubated for 4 minutes at 4° C., pelleted, resuspended in 1× PERM with PANEL 3 (Table 1) cytokine antibodies, incubated 30 minutes at 4° C., washed with 200 μL Perm, and washed with 200 μL FACS.

TABLE 1
Staining Panels

| | PANEL 1A- | PANEL 1B - | PANEL 2 - | PANEL 3- |
|---|---|---|---|---|
| Color | Naïve | Naive | Proliferation | Cytokine |
| BUV395 | CD8 | CD8 | CD8 | CD8 |
| E450 | CD4 | CD130 | CD4 | CD4 |
| APC/e660 | tbet | Mir-130 | CD44 | IFNy |
| APC e780 | Viability | Viability | Viability | Viability |
| FITC | Mir-29 | CD4 | CFSE | |
| PerCP e710 | CD44 | CD44 | | CD44 |
| PE | CD122 | IRF1 | CD25 | TNFa |
| PETxR/ | eomes | CD62L | CD62L | GranB |
| PEe610 | | | | |
| PECy7 | CD62L | CD122 | CD69 | CD62L |

Sequencing Sort. Cells were prepared for sequencing as follows: (1) Resuspend remaining pellet in antibody cocktail: PANEL: 5 ul→CD8 (e450)+CD4 (FITC)+Viability Dye (APCe780); (2) incubate for 30 min at 4° C.; (3) wash 1× with 5 ml MACs. Centrifuge at 500 g for 5 minutes; (4) aspirate supernatant after wash; (5) make single color bead controls; (6) make collection tubes: 2-3 eppendorf tubes w/100 ul sort collection buffer; (7) resuspend final pellet in 200 ul MACS; (8) place in top of blue mesh-capped flow tubes and centrifuge at 500 g for 1 min to filter final sample; (9) pipette up/down to resuspend pellet; (10) Sort: 100K for RNASeq—CD8+CD4− Viabilitylo mir29+mir130+; (11) spin down sorted cells; (12) resuspend pellet in 1 ml Trizol and let sit at RT for 5 minutes; and (13) store at −80° C.

Nanoprojection Conditions. Silicon dioxide etching mask etching condition: ‘CHF3/O2 Oxide Etching’—etching rate: 150 nm/min; etching process 3 mins (for silicon oxide). Silicon etching (deep silicon etching): ‘HBr/Ar-Oxide-1’—Etching rate: 156 nm/min, silicon to oxide: 27:1; etching process 18 minutes. Tapering etching: ‘CF4 Etching’—Etching rate: 71 nm/min; for piece of wafers—3 minutes 45 seconds; for whole wafers—22 minutes.

### Example 1—Nanoprojection Array Fabrication

A an approximately 3000 Å thick silicon dioxide (SiO₂) layer was deposited onto a silicon wafer by wet oxide annealing (FIG. 1A). Fine patterns of nanoprojection arrays were developed using deep UV photolithography (FIG. 1B). To prepare deep silicon etching for nanoprojection structure fabrications, a fine pattern was transferred to the oxide layer via dry etching (FIG. 1C). Along with inductively coupled plasma, the length of the nanoprojection structures varied with etching time and thickness of the oxide mask, making a higher aspect ratio nanoprojections (FIG. 1D; FIG. 2A). To achieve more delivery efficacy and a cell-friendly environment, the tapering process of nanoprojection structures was proceeded by using a soft dry etching process (FIG. 1E; FIG. 2B). After obtaining nanoprojections with sub-10 nm tips, the silicon surface was functionalized with strongly charged capturing layers.

To obtain a positive surface charge for biomolecule complexation, the silicon nanoprojection surface was functionalized with N-hydroxysulfosuccinimide (NHS) moieties, which were then conjugated with polyethyleneimine (PEI, branched, 25 kDa) (FIG. 1F-1G). To obtain a negative surface charge for biomolecule complexation, 3-(trihydroxysilyl)-1-propanesulfon was covalently conjugated to bring in the negatively charged sulfonate moieties. After shipping the target biomolecules through the dry coating, target cells were cultured on the nanoprojection devices to induce the intracellular delivery (FIGS. 1H-1I; FIG. 2C).

### Example 2—Bare Nanoprojection Arrays do not Effectively Deliver Biomolecules to Target Cells

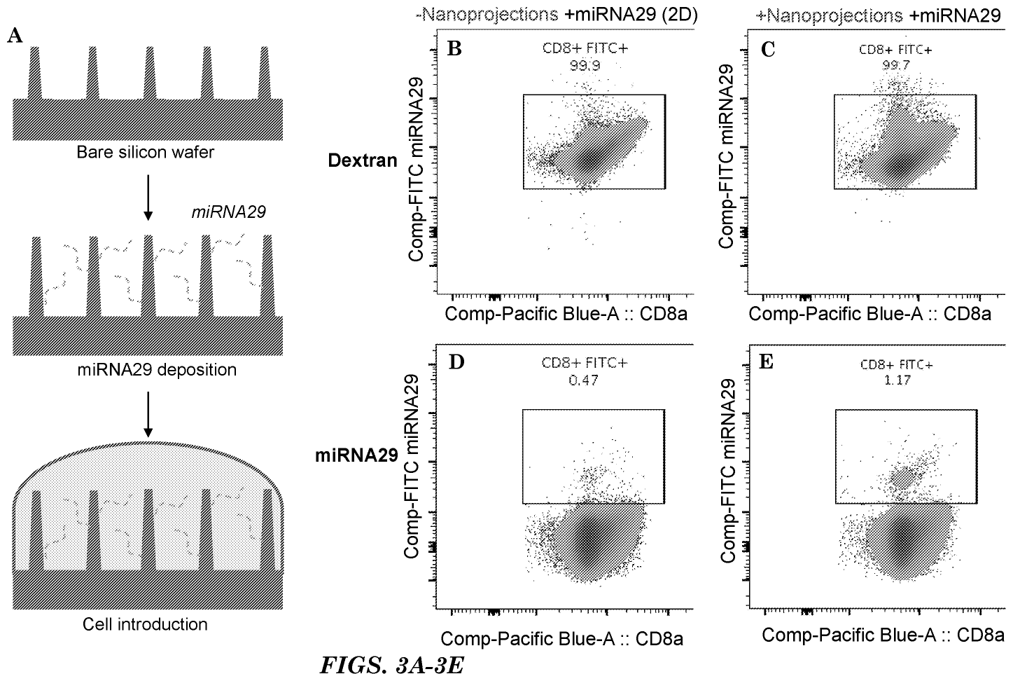

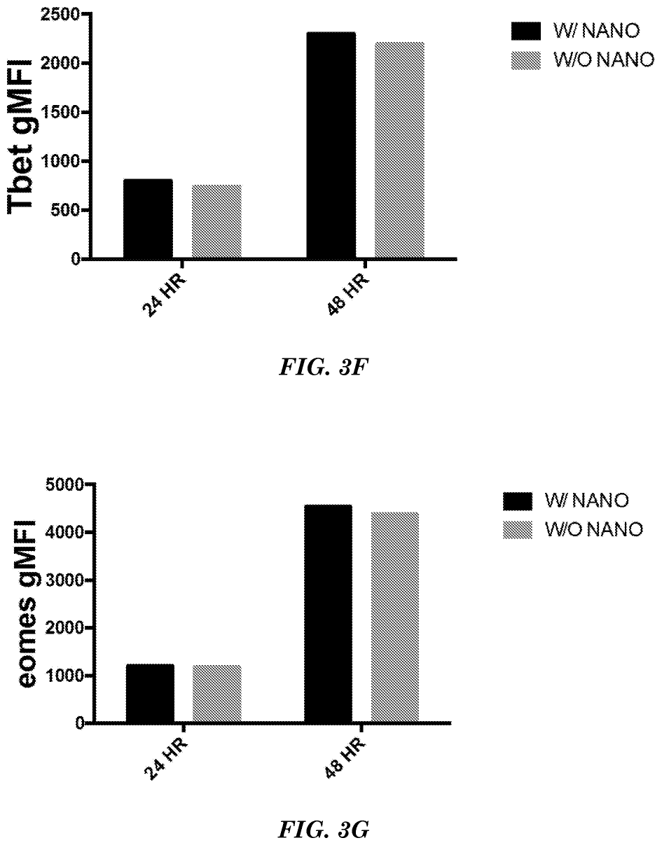

To test the delivery efficiency of bare silicon nanoprojection structures, bare silicon nanoprojection arrays were coated with FITC-Dextran (3,000-5,000 g/mol) or miRNA (13,885 g/mol) (FIG. 3A). FITC-Dextran entered the intercellular region of CD8⁺ T cells cultured with either FITC-Dextran alone (FIG. 3B) or a FITC-Dextran-coated nanoprojection array (FIG. 3C). However, miRNA barely penetrated the CD8⁺ T cell membrane (FIG. 3D), even when T cells were cultured in the presence of a miRNA-coated nanoprojection array (FIG. 3E). To determine whether nanoprojection-mediated delivery of miRNA29-FITC reduces the expression of its target genes (e.g., Eomesodermin (EOMES)) in a predictive manner, flow cytometry was performed on CD8⁺ T cells after 24 hours and 48 hours of culture with bare nanoprojection arrays. No significant changes in transcription factors were observed when RNA was deposited onto bare nanoprojection arrays (FIGS. 3F-3G).

### Example 3—Modified Nanoprojection Arrays Demonstrate Improved Biomolecule Delivery to Target Cells

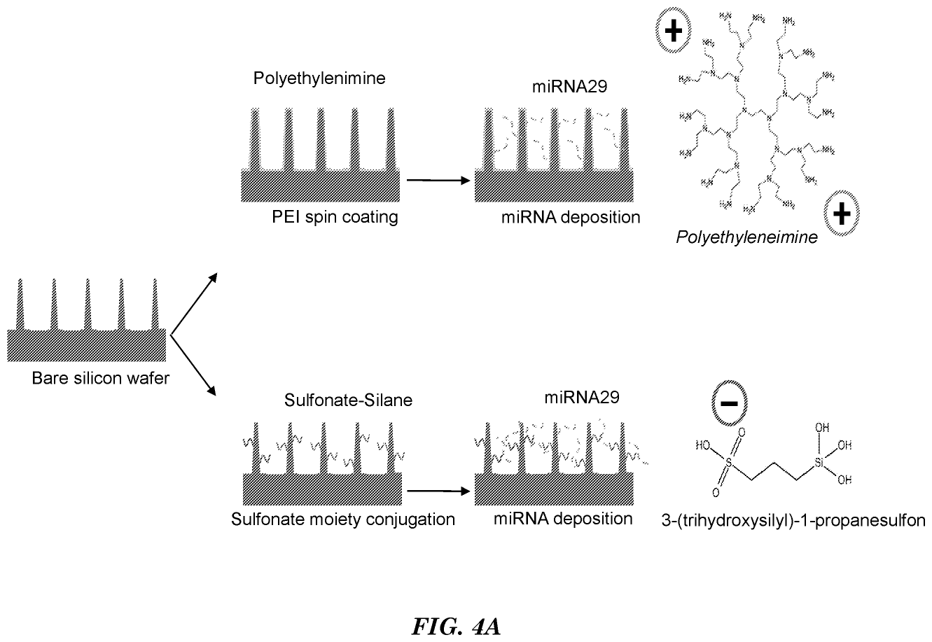

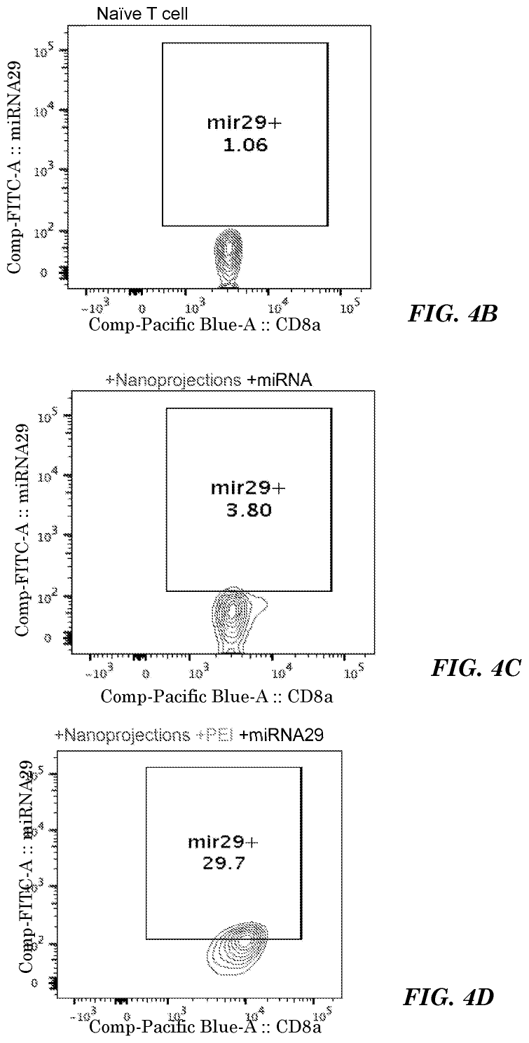

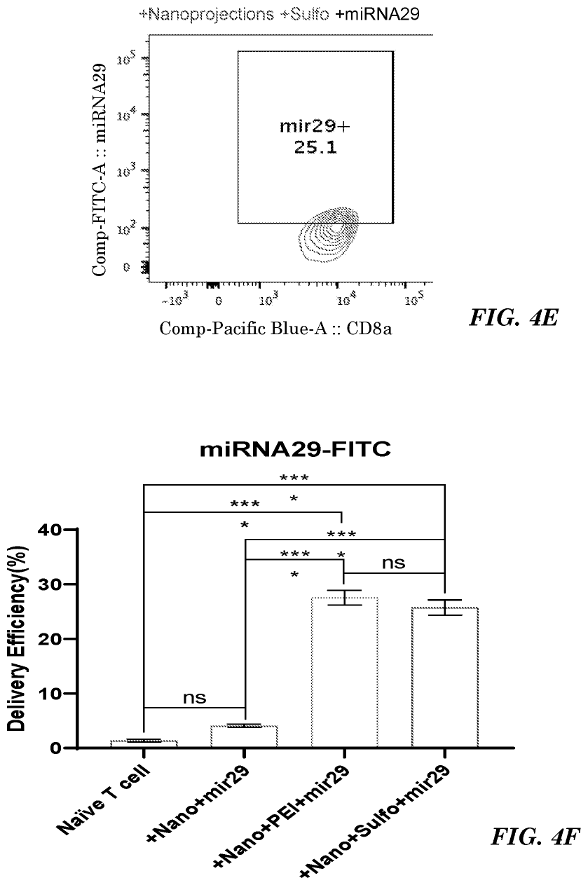

To increase the delivery efficiency, the silicon nanoprojection surface was modified with strongly charged capturing layers. To generate positively charged nanoprojections, silicon nanoprojection arrays were spin-coated (3,000 rpm, 1 minute) with polyethyleneimine (PEI) and then miRNA was deposited onto the coating surface (FIG. 4A, top panel).

To generate negatively charged nanoprojection arrays, 3-(trihydroxysilyl)-1-propanesulfon was introduced with a vapor phage coating method to bring in the negatively charged sulfonate moieties (FIG. 4A, lower panel). Compared with solute miRNA and bare silicon nanoprojection structures, flow cytometry data of PEI and sulfonate coated functionalized nanoprojection structures show higher delivery efficiency (˜25 percent) (FIGS. 4B-4F).

### Example 4—Dose-Dependent Biomolecule Delivery to Target Cells

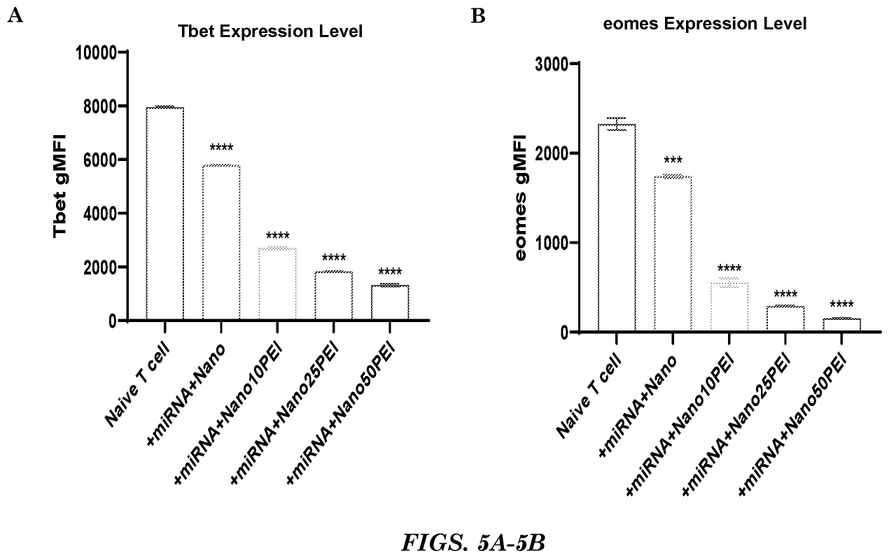

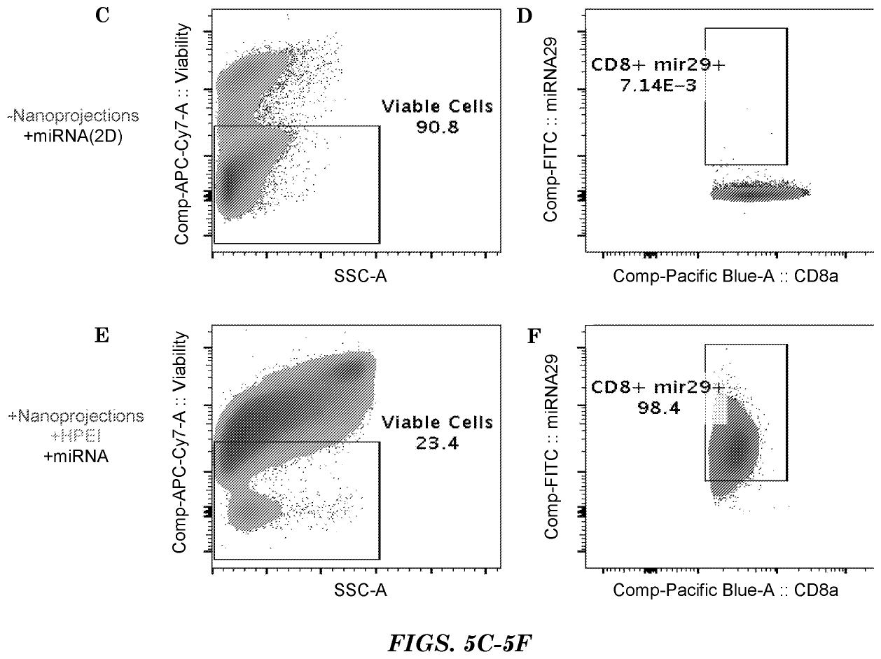

PEI has an advantage of controllability over the level of the transfection by adjusting the weight percent of the coating solution (FIGS. 5A-5B). By increasing the concentration of the PEI solution, the number of delivered miRNA can be enhanced and the target gene expression is significantly down-regulated. However, PEI causes a cytotoxic effect through either the disruption of the cell membrane (immediate) or disruption of the mitochondrial membrane after internalization (delayed) (FIG. 5E), even though it shows a high transmission (˜95 percent) efficiency (FIG. 5F).

### Example 5—Silicon Nanoprojection Arrays Comprising NHS-PEG-Silane Linkers Demonstrate Improved Biomolecule Delivery to Target Cells with Reduced Toxicity

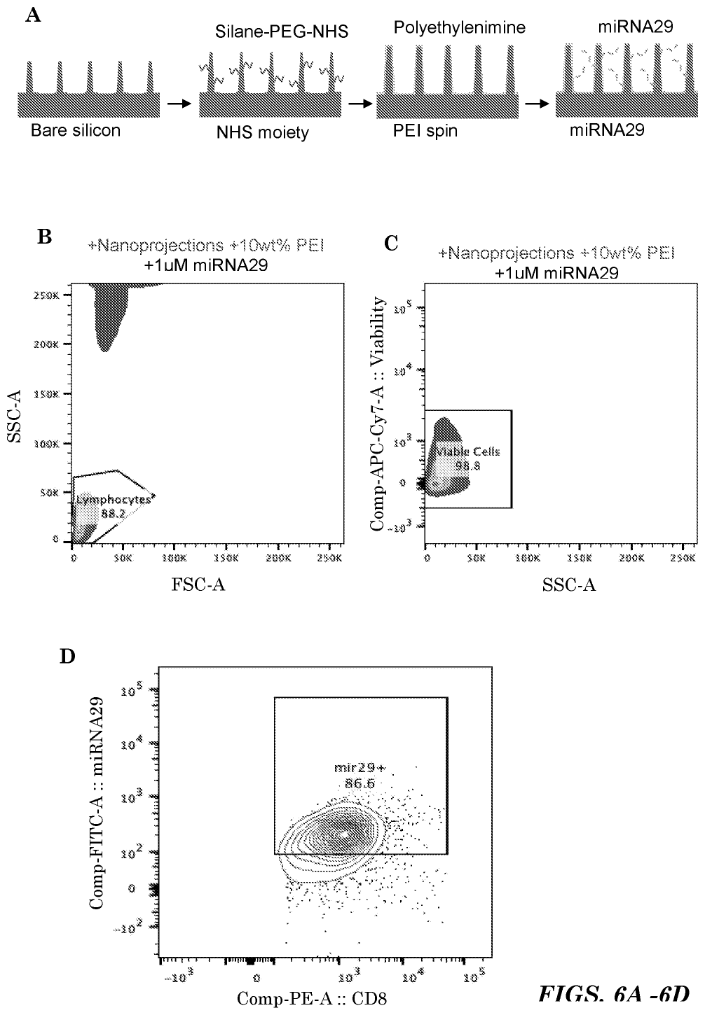

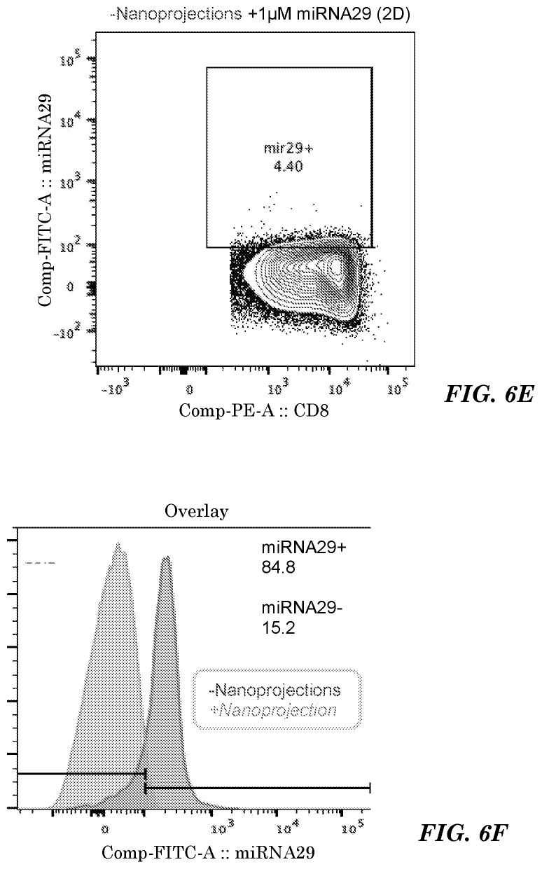

To reduce the degradation of PEI and cell toxicity, a covalent crosslinker, NHS-PEG-silane, was used to anchor PEI to the silicon surface (FIG. 6A). The NHS moieties are easily able to make an amide conjugated with the primary amine on the PEI chains. By providing a minimal contact PEI to the cells and fully covered silicon surface with branched-chain, cell viability was increased to over 95 percent and delivery efficiency was increased to over 85 percent (FIGS. 6C-6D).

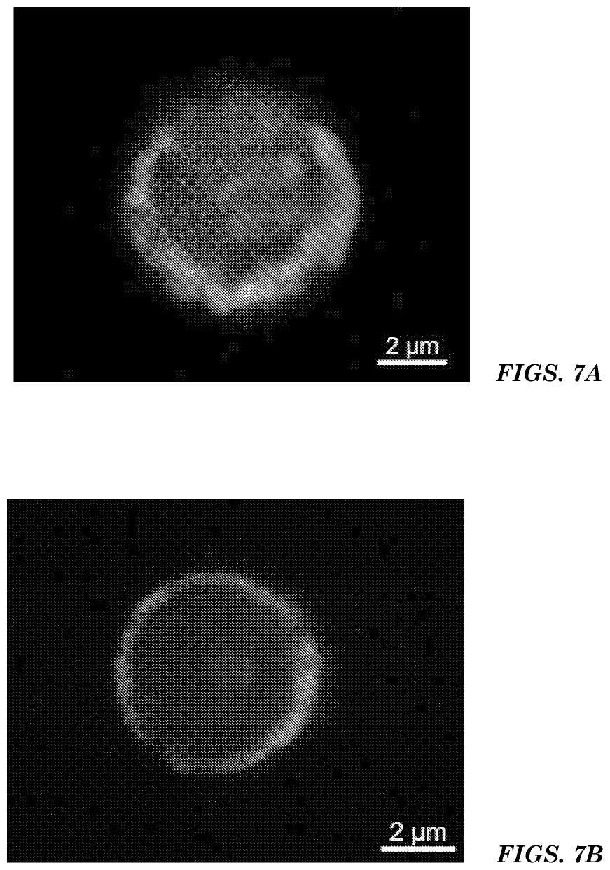

Confocal microscopy images confirmed the intracellular transportation of RNA-fluorophores (FIG. 7A-7B). Both inert RNA and miRNA conjugated with FITC show the fluorescence signals overall in the cytoplasmic area.

### Example 6—Dose Response Studies—RNA

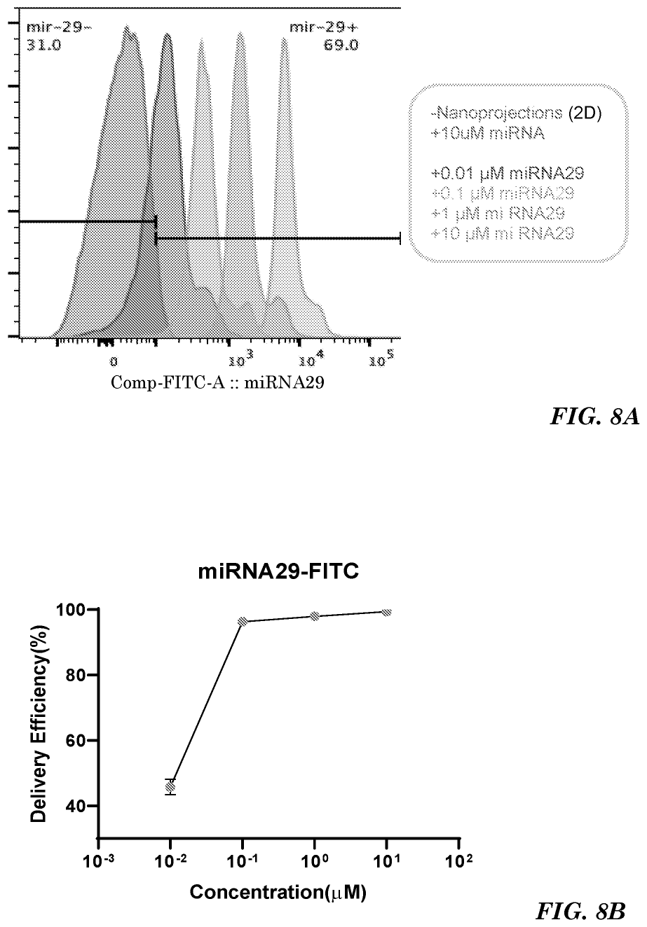

Next, the effect of using different concentrations of initial RNA was evaluated. FIG. 8B shows a trend of the saturation curve over 100 nM initial loading. With this result, the optimal loading concentration compatible with the PEI conjugation density and silicon surface area was confirmed.

### Example 7—Dose Response Studies—miRNA

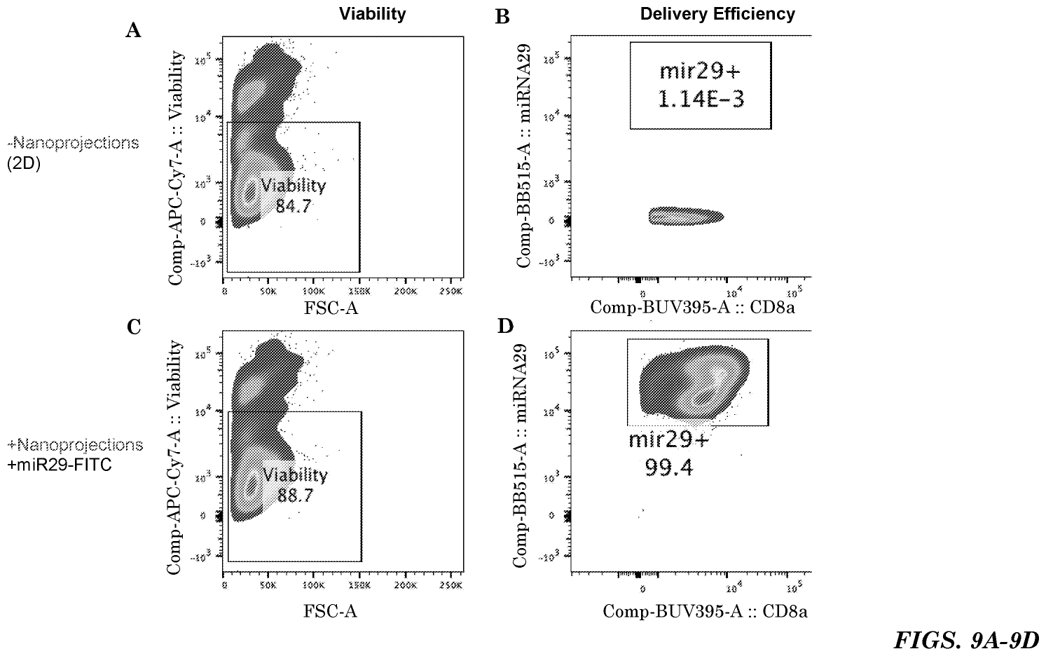

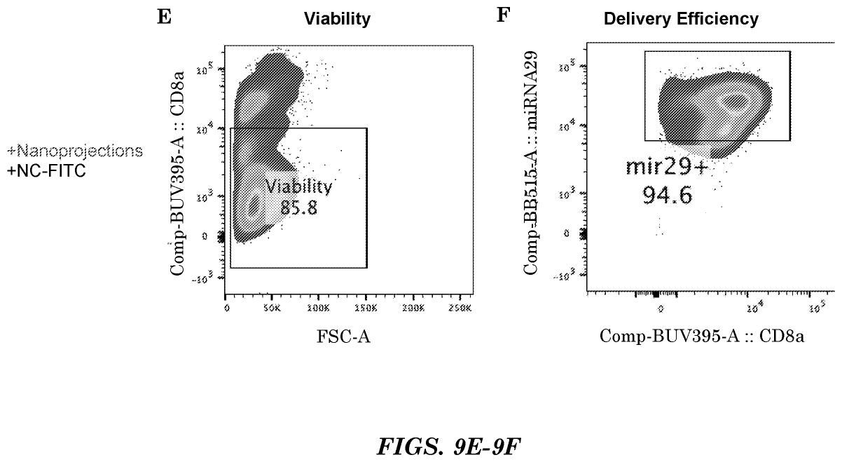

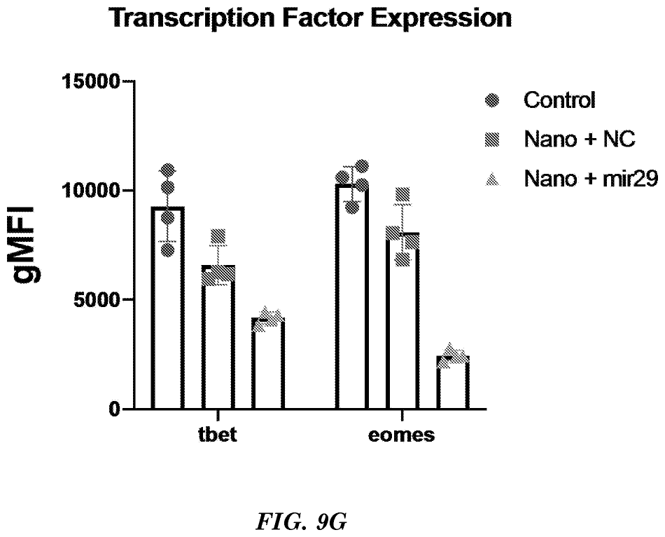

To confirm the efficacy of miRNA29 to their target transcription factors and delivery rates of similar biomolecules, inertRNAs (NC, negative control) were inserted for the comparison. Both of inert RNA and miRNA29 showed high cell viabilities and delivery efficiency (FIGS. 9C-9F). Also, target expression levels of TBET and EOMES were highly down-regulated with miRNA29 (FIG. 9G).

### Example 8—Modification of Adult CD8⁺ T Cells

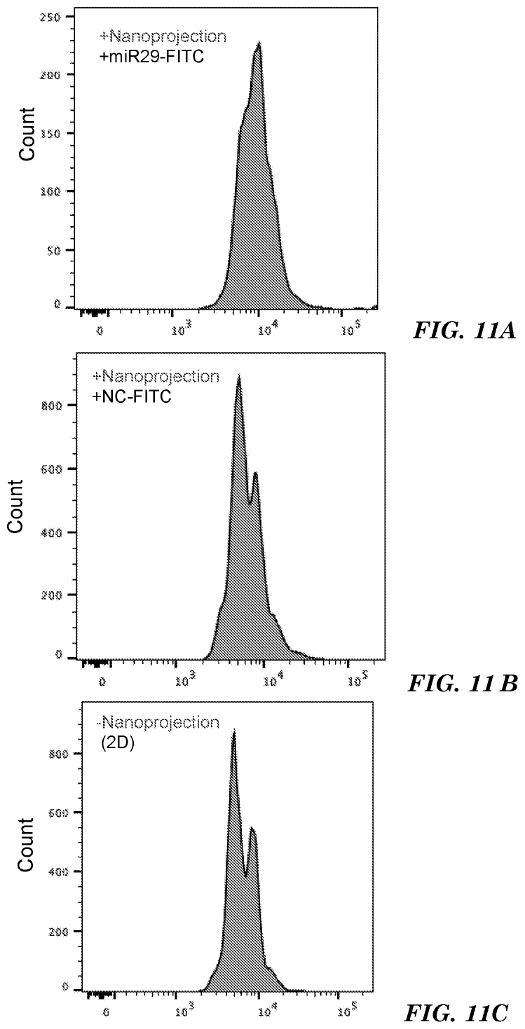

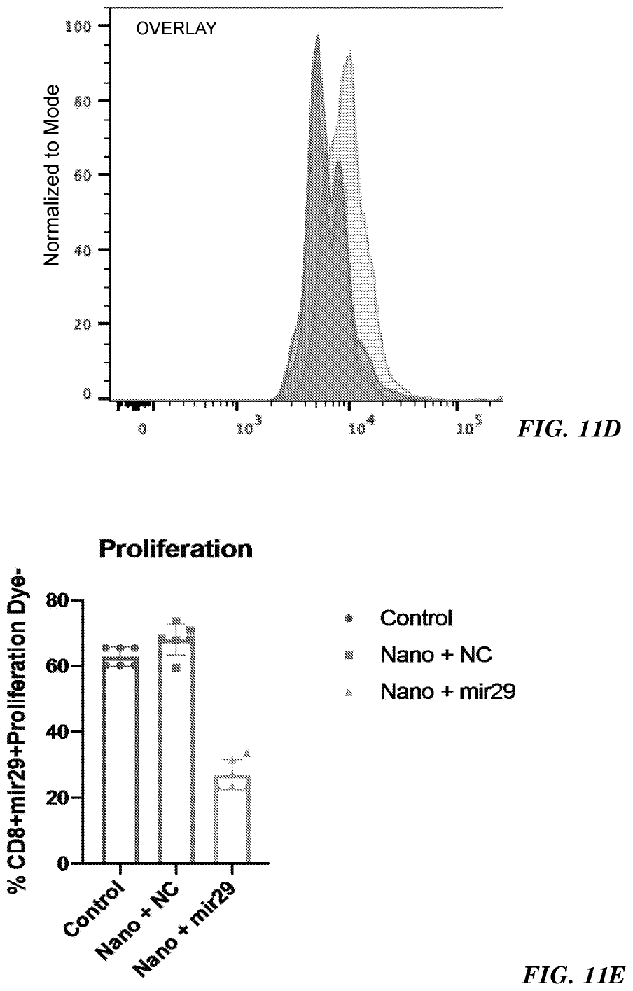

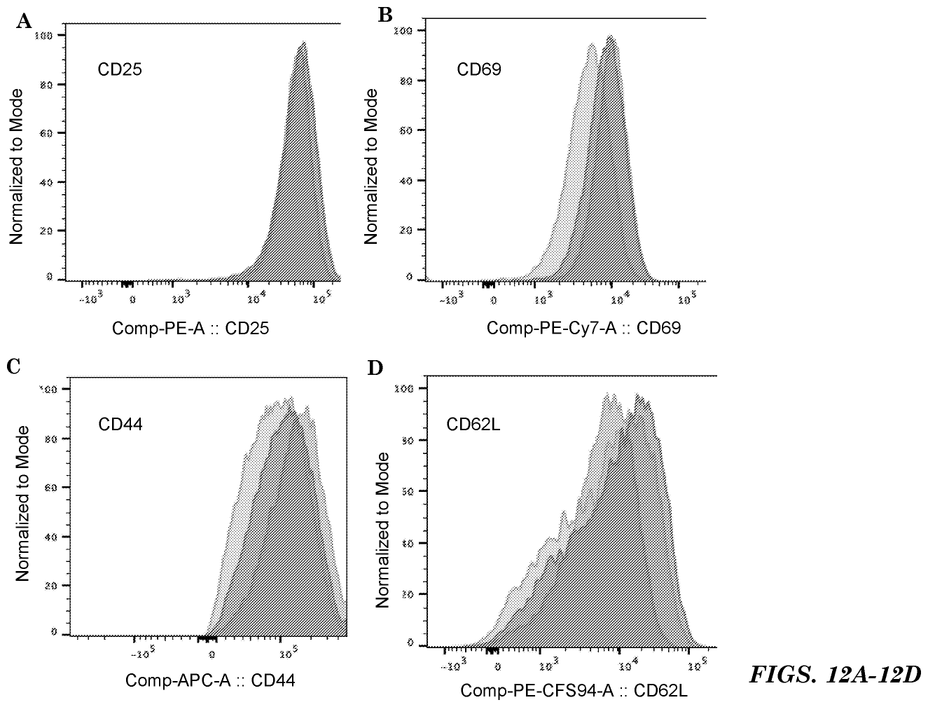

To test altered function of nanoprojection modified adult CD8⁺ T cells following single delivery of a mir-29 mimic, three outputs of T cell function were assessed after T cell receptor stimulation—Proliferation, Activation and Cytokine Production. Murine Proliferation Dye coated nanoprojection modified splenic CD8+ T cells showed that delivery of mir-29 mimic reduced proliferative capacity after antigen stimulation at 48 hours (FIGS. 11A-11E). This is observed as decreased dilution of the proliferation dye by ˜55% compared to the controls (negative control scrambled sequence delivery ˜NC (middle bar) and cells only (left bar) (FIG. 11E).

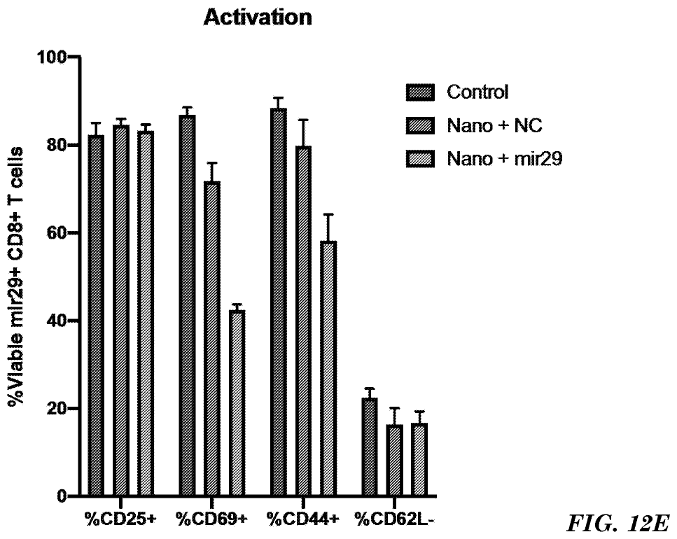

Nanoprojection delivery of mir-29 mimic also downregulated early (CD69) and late (CD44) activation markers at 48 hours by 30% and 10%, respectively following antigenic TCR stimulation but no difference in differentiation marker CD62L or early activation marker CD25 possibly because the cells all upregulated CD25 (IL-2R) after incubating in media with IL-2 (FIGS. 12A-12E).

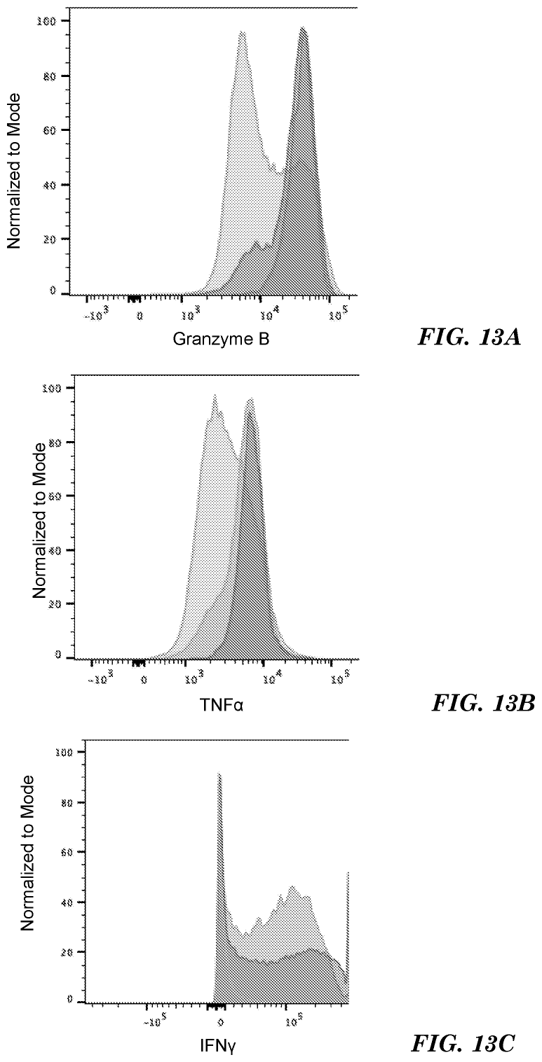

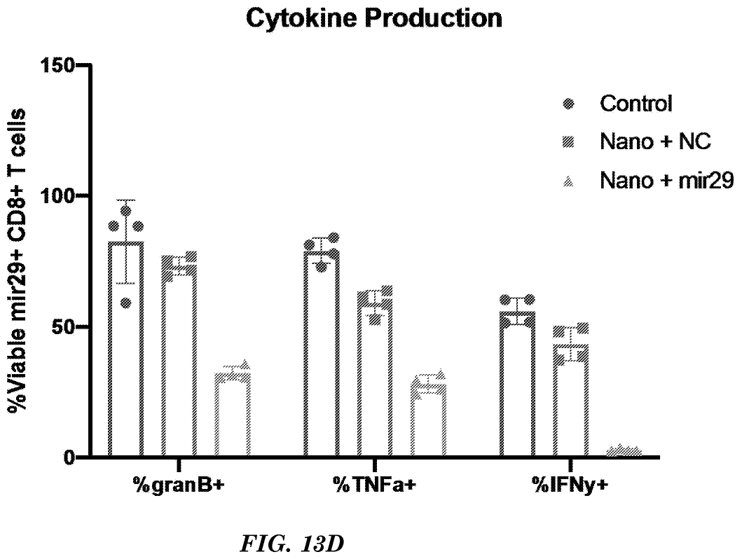

After 24 hours of antigen stimulation and 5-hour brefeldin A incubation, nanoprojection-mir29 modified CD8⁺ T cells showed significant decrease in cytolytic molecule production—granzyme B, TNFα and IFNγ—compared to NC and cell only controls (FIGS. 13A-13D). These findings suggest that nanoprojection single delivery can overexpress mir-29 in naïve CD8⁺ T cells to ultimately reduce proliferative capacity, activation capacity and pro-inflammatory cytokine secretion compared to NC and cell only controls.

### Example 9—Co-Delivery Studies

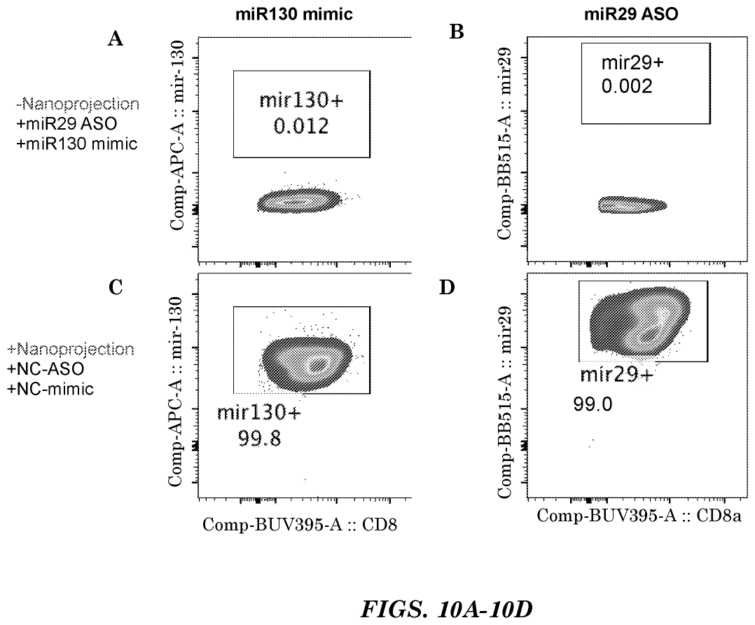

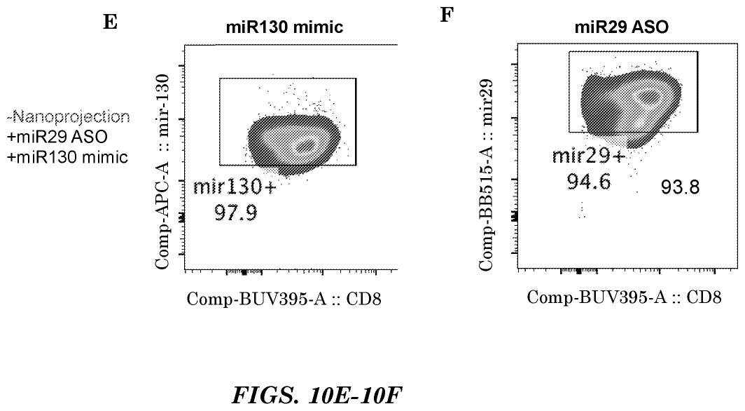

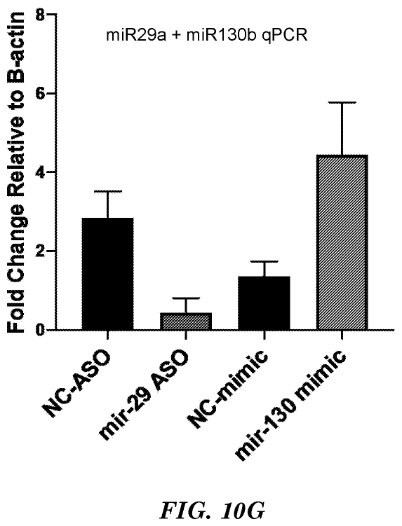

Co-delivery of microRNAs into cells is an attractive strategy for synergetic effects of desirable change in the target expression. To test that, the co-delivery efficiency of two different miRNAs was evaluated to determine which may boost their transcriptional effects. Negative controls of both antisense oligonucleotides (ASO) and mimic showed over 99 percent delivery efficiency (FIGS. 10C-10D). The effector miRNAs, miRNA29 ASO, and miRNA130 mimic, also displayed good transmission levels (FIGS. 10E-10F). As assessed by miRNA qPCR at 36 hours, the fold change level of mir-29 was significantly reduced and the fold change level of mir-130 was significantly increased compared the negative control (relative to the b-actin housekeeping gene) (FIG. 10G), which suggests that the nanoprojection platform can efficiently deliver genetic material to knockdown and overexpress gene expression simultaneously.

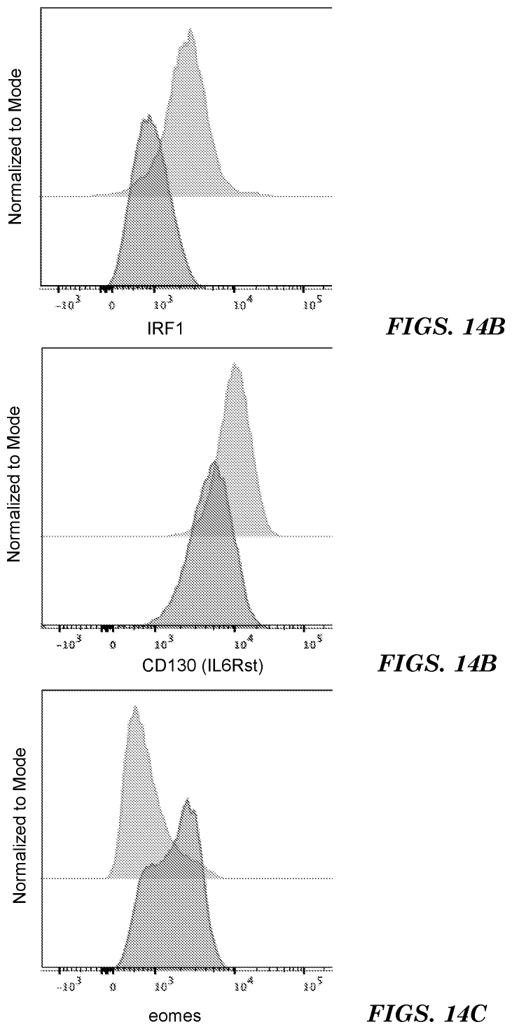

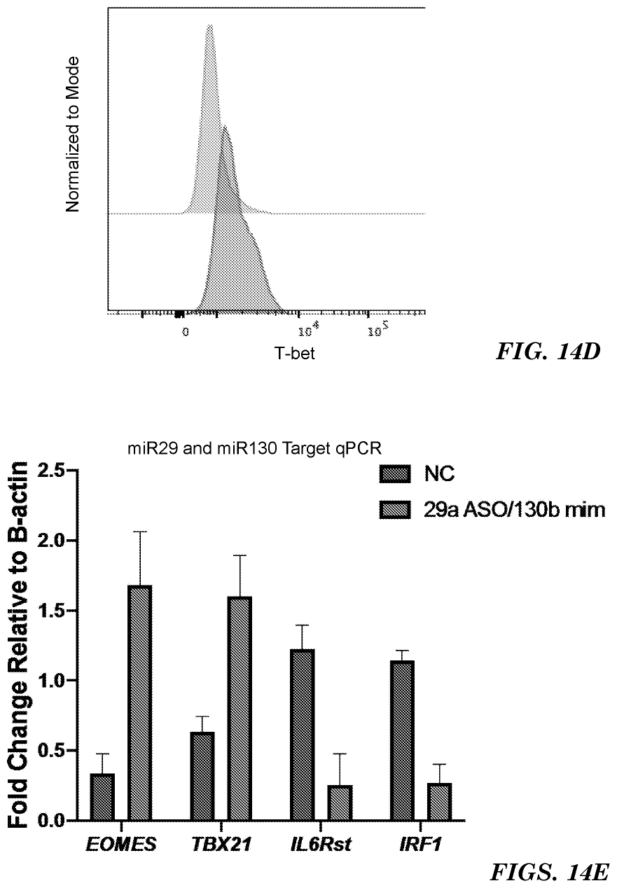

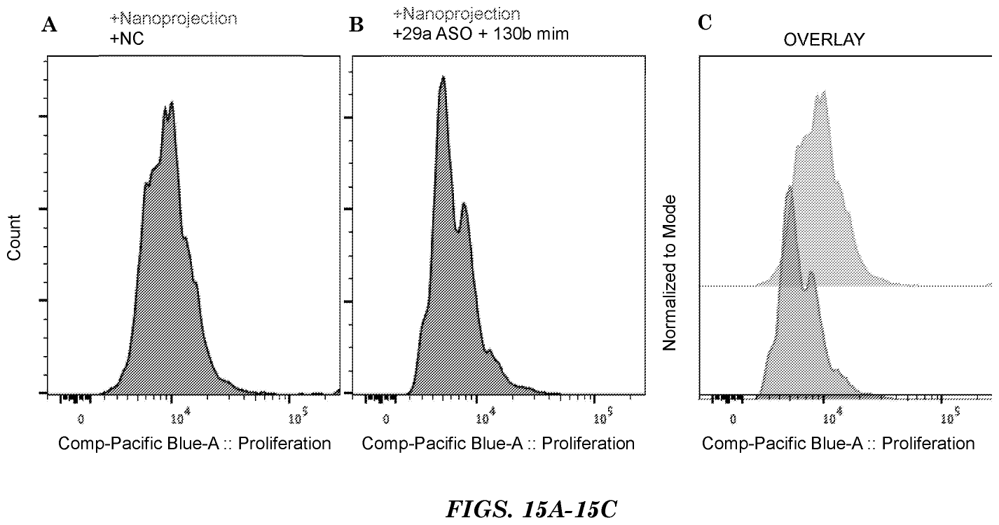

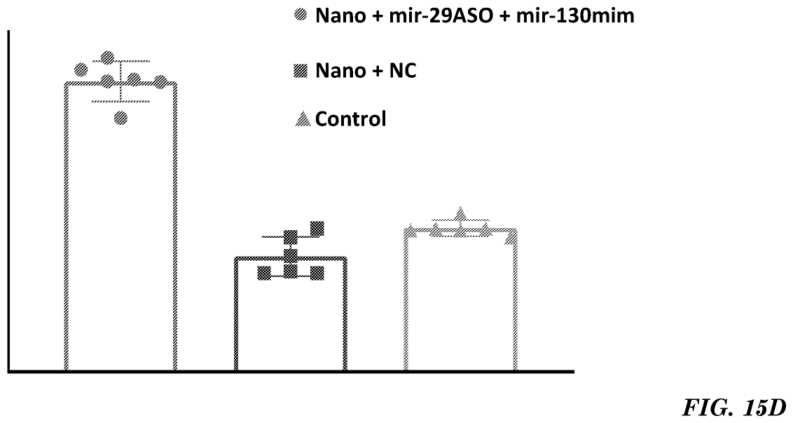

Next, whether toggling mir-29 and mir-130 levels has an effect on downstream direct target expression on both an RNA and protein level was assessed. mir-130 was observed to downregulate IRF1 and CD130, as compared the negative control after nanoprojection delivery of mir-130 mimic on the protein level (FIGS. 14A-14B) and RNA level (relative to b-actin housekeeping gene) (FIG. 14E). mir-29 was also observed to target T-bet and EOMES, which were upregulated compared the negative control after nanoprojection delivery of mir-29 antagomir on the protein level (FIGS. 14C-14D) and RNA level (FIG. 15E).

To test altered function of nanoprojection modified adult CD8⁺ T cells following co-delivery of a mir-130 mimic and mir-29 antagomir (ASO), proliferation, activation, and cytokine production was assessed. Murine proliferation dye coated nanoprojection modified splenic CD8⁺ T cells showed that co-delivery of mir-29 antagomir and mir-130 mimic, significantly increased proliferative capacity after antigen stimulation at 48 hours. This is observed as increased dilution of the proliferation dye by ˜45% compared to the controls (negative control scrambled sequence delivery—NC [blue] and cells only [orange] (FIGS. 15A-15D).

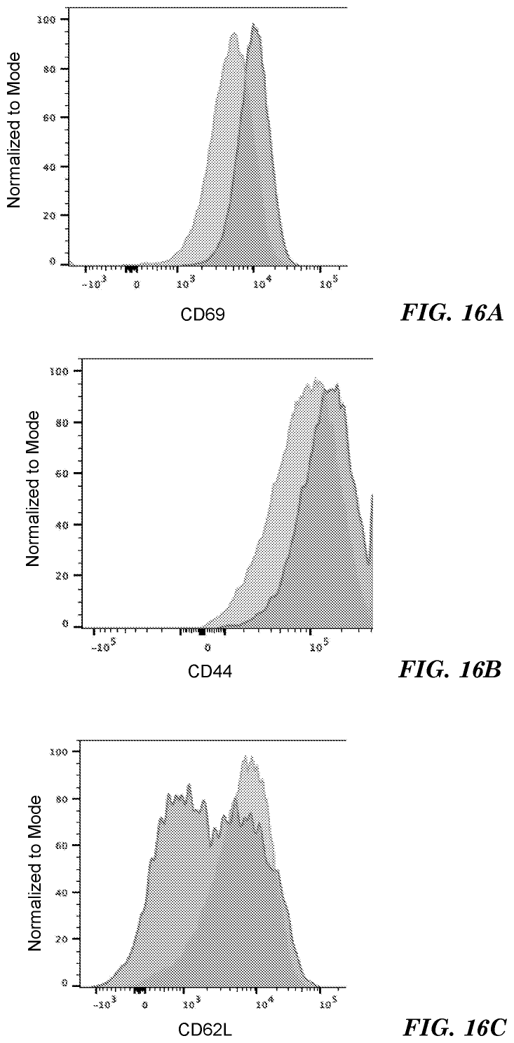

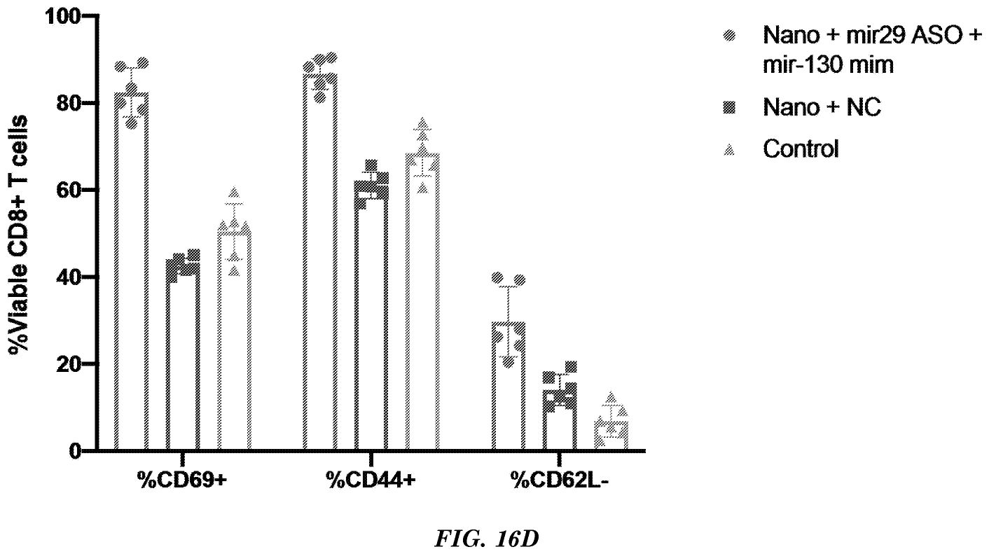

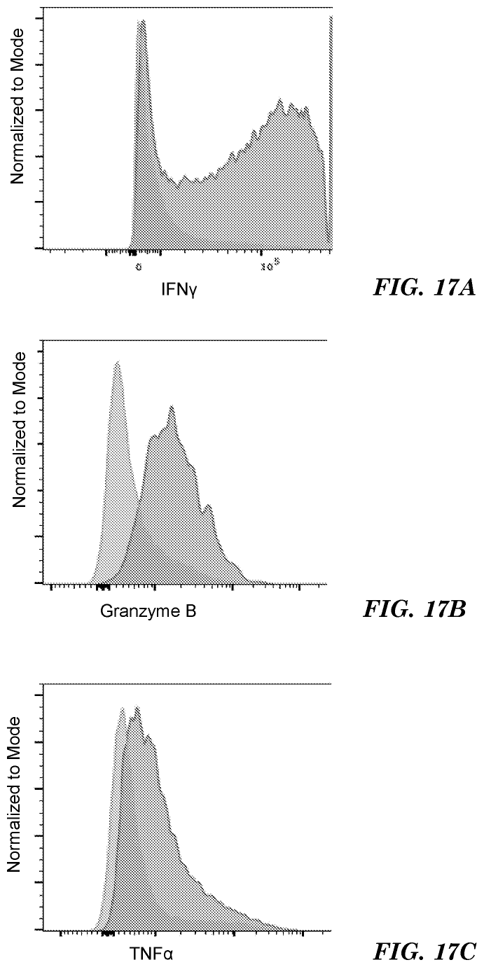

Nanoprojection co-delivery of mir-29 ASO and mir-130 mimic also highly upregulated early (CD69) and late (CD44) activation markers at 48 hours by ˜40% and ˜20% respectively compared to the control and also downregulated differentiation marker CD62L by ˜10% compared to the control which suggests that nanoprojection co-modified CD8+ T cells induce a highly activated and differentiated state after TCR stimulation (FIGS. 16A-16D).

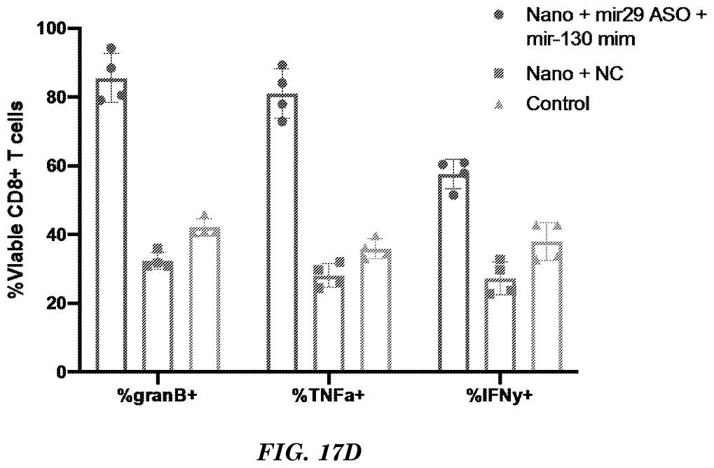

After 24 hours of antigen stimulation and 5-hour brefeldin A incubation, nanoprojection-mir29/mir-130 co-modified adult CD8⁺ T cells showed significant increase in cytolytic molecule production—granzyme B, TNFα and IFNγ—by as high as ˜50% compared to NC and cell only controls (FIGS. 17A-17D). These findings suggest that nanoprojection co-delivery can overexpress mir-130 and knockdown mir-29 in naïve CD8+ T cells to ultimately increase proliferative capacity, activation capacity and pro-inflammatory cytokine secretion after TCR stimulation compared to NC and cell only controls.

### Discussion of Examples 1-9

The use of nanoprojection platform to perturb target cells represents a promising, minimally invasive strategy because it allows for effector specific manipulation with a negligible effect on cell survival and functioning. Furthermore, the effective delivery of cell effectors can regulate cellular behavior, expressing desired phenotypes, and activating to express specific markers. In the future, by combining with mass production and the scalable ability of nanoprojection devices, this platform might allow the manufacture patient-specific combinatorial therapies in the company with sustainable manipulation of high-quality activated immune cells at large-scale.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed is:

1. A silicon nanoprojection device comprising:
a substrate having a surface and
one or more nanoprojection structures having a proximal end attached to said substrate and extending away from the surface of the substrate to a distal end, wherein said one or more nanoprojection structures have a configuration which tapers narrowingly from the proximal end to the distal end.

2. The silicon nanoprojection device according to claim 1 further comprising:
an ionic coating over said one or more nanoprojection structures.

3. A silicon nanoprojection device comprising:
a substrate having a surface;
one or more nanoprojection structures having a proximal end attached to said substrate and extending away from the surface of the substrate to a distal end; and
an ionic coating on said one or more nanoprojection structures.

4. The silicon nanoprojection device of claim 1 or 3, wherein the proximal end has a cross-sectional diameter of 10-500 nm and the distal end has a cross sectional diameter of 1-200 nm.

5. The silicon nanoprojection device of claim 1 or 3, wherein the one or more nanoprojection structures have a length of 0.5-20 μm.

6. The silicon nanoprojection device of claim 1 or 3, wherein the silicon nanoprojection device has an array of nanoprojection structures.

7. The silicon nanoprojection device of claim 1 or 3, wherein the one or more nanoprojection structures are spaced 1-100 μm apart on the surface of said substrate.

8. The silicon nanoprojection device of claim 1 or 3, wherein a surface of the one or more nanoprojection structures is covalently modified with a modifier.

9. The silicon nanoprojection device of claim 8, wherein the modifier is selected from the group consisting of N-hydroxysulfosuccinimide (NHS), polyethylene glycol (PEG), 3-(trihydroxy-silyl)-1 propanesulfon, and silane.

10. The silicon nanoprojection device of claim 2 or 3, wherein said ionic coating is a cationic polymer selected from the group consisting of polyethyleneimine (PEI), poly-1-lysine (PLL), chitosan, and combinations thereof.

11. The silicon nanoprojection device of claim 2 or 3, wherein said ionic coating is bonded to or interacting with a modifier, wherein, the modifier is on the surface of the one or more nanoprojection structures.

12. The silicon nanoprojection device of claim 2 or 3, wherein said ionic coating is an anionic polymer selected from the group consisting of sulfonyl, carboxyl, phosphate, alkoxide, and combinations thereof.

13. The silicon nanoprojection device of claim 2 or 3 further comprising:
a biomolecule complexed to said ionic coating.

14. The silicon nanoprojection device of claim 13, wherein the biomolecule is selected from the group consisting of a nucleic acid molecule, a protein or peptide fragment, a carbohydrate, a small molecule, and a combination thereof.

15. The silicon nanoprojection device of claim 14, wherein the biomolecule is a nucleic acid molecule selected from the group consisting of an RNA molecule, an DNA molecule, and an aptamer.

16. The silicon nanoprojection device of claim 15, wherein the biomolecule is an RNA molecule selected from the group consisting of a small interfering RNA (siRNA) molecule, a short or small hairpin RNA (shRNA) molecule, a micro RNA (miRNA) molecule, a messenger RNA (mRNA), an antisense oligonucleotide (ASO) and a ribozyme.

17. The silicon nanoprojection device of claim 15, wherein the biomolecule is a DNA molecule selected from the group consisting of a vector or a plasmid.

18. The silicon nanoprojection device of claim 14, wherein the biomolecule is a protein selected from the group consisting of a cytokine, a chemokine, a toxin, an antibody, an agonist, an inhibitor, a transcription factor, a protease, an enzyme, and a receptor.

19. The silicon nanoprojection device of claim 14, wherein the biomolecule is a small molecule selected from the group consisting of a dye, a quantum dot, and a nanoparticle.

20. The silicon nanoprojection device of any one of claims 13 to 19 further comprising:
one or more target cells into which the one or more nanoprojection structures extends.

21. The silicon nanoprojection device of claim 20, wherein the one or more target cells are animal cells.

22. The silicon nanoprojection device of claim 21, wherein the animal cells are mammalian cells.

23. The silicon nanoprojection device of claim 22, wherein the mammalian cells are human cells.

24. The silicon nanoprojection device of claim 23, wherein the human cells are primary cells.

25. The silicon nanoprojection device of claim 20, wherein the one or more target cells are bacterial cells.

26. The silicon nanoprojection device of claim 20, wherein the one or more target cells are plant cells.

27. A pair of silicon nanoprojection devices of any one of claims 20 to 26 between the substrates of which the one or more target cells are sandwiched.

28. A method of making a nanoprojection device, said method comprising:
providing a silicon monolithic structure and
carrying out a series of nanofabrication steps on the silicon monolithic structure to form one or more nanoprojection structures having a proximal end attached to a surface of a substrate and extending away from the surface of the substrate to a distal end, wherein said one or more nanoprojection structures have a configuration which tapers narrowingly from the proximal end to the distal end.

29. The method according to claim 28, wherein said carrying out a series of nanofabrication steps comprises:
depositing an etching mask layer onto the silicon monolithic structure;
coating the deposited etching mask layer with a resist layer;
patterning the silicon monolithic structure with the resist coated mask layer, using lithography, to produce, upon development, one or more nanoprojection structures extending from a surface;
developing the patterned silicon monolithic structure with the coated mask layer into the one or more nanoprojection structures extending from the surface using mask etching and deep silicon reactive-ion etching (RIE); and
tapering the nanoprojection structures using tapered etching.

30. The method of claim 29, wherein the etching mask layer is a silicon dioxide layer, a polymer layer, or a metal layer.

31. The method of claim 30, wherein the etching mask layer is silicon dioxide and said depositing is carried out by dry oxide annealing or wet oxide annealing.

32. The method of claim 31, wherein said developing the patterned silicon monolithic structure is carried out by silicon oxide mask etching.

33. The method of claim 29, wherein said developing the patterned silicon monolithic structure is carried out to remove the etching mask layer.

34. The method of any one of claims 29 to 33 further comprising:
covalently modifying a surface of the one or more nanoprojection structures with a modifier.

35. The method of claim 34, further comprising:
conjugating a polymer to the modified one or more nanoprojection structures.

36. The method of claim 35, wherein the polymer is a cationic polymer.

37. The method of claim 35, wherein the polymer is an anionic polymer.

38. The method of any one of claims 36 to 37 further comprising:
complexing a biomolecule on the one or more modified, polymer coated nanoprojection structures.

39. The method of claim 38, wherein the biomolecule is selected from the group consisting of a nucleic acid molecule, a protein or peptide fragment, a carbohydrate, a small molecule, and a combination thereof.

40. A method for delivering a biomolecule to a target cell, the method comprising:
providing a silicon nanoprojection device according to any one of claims 12 to 18 and
contacting one or more target cells with the one or more nanoprojection structures of the silicon nanoprojection device so that the one or more nanoprojection structures extend into the one or more target cells.

41. The method of claim 40 further comprising:
centrifuging the silicon nanoprojection device during said contacting to deliver the biomolecule into the target cell.

42. The method of any one of claims 40 to 41, wherein the one or more target cells are animal cells.

43. The method of claim 42, wherein the animal cells are mammalian cells.

44. The method of claim 43, wherein the mammalian cells are human cells.

45. The method of claim 44, wherein the human cells are primary cells.

46. The method of any one of claims 40 to 41, wherein the one or more target cells are bacterial cells.

47. The method of any one of claims 40 to 41, wherein the one or more target cells are plant cells.

48. The method of any one of claims 40 to 47, further comprising:
providing a second one of the silicon nanoprojection device having one or more nanoprojection structures complexed with a biomolecule and
contacting the one or more target cells with the second one of the silicon nanoprojection device to form a sandwich structure of the one or more target cells between the first and second silicon nanoprojection devices.

49. One or more modified target cells produced according to the method of any one of claims 40 to 48.

50. A method of treating a subject with a modified cell, said method comprising:
selecting a subject in need of treatment with a modified cell and
administering the one or more modified target cells of claim 48 to the selected subject.

51. The method of claim 50, wherein the subject is a mammalian subject.

52. The method of claim 46, wherein the subject is a human subject.

53. The method of any one of claims 50 to 52, wherein the subject is suffering from a disease or disorder.

54. The method of claim 53, wherein the disease or disorder is a cancer.

55. The method of claim 54, wherein the target cell is a primary cell.

56. The method of claim 55, wherein the primary cell is a lymphocyte.

57. The method of claim 56, wherein the lymphocyte is a T cell or a B cell.

58. The method of claim 50, wherein the subject is a plant.
